(19) United States
(12) Patent Application Publication
(10) Pub. No.: US 2025/0012286 A1
(43) Pub. Date: Jan. 9, 2025

(54) PORTABLE WIND BLOWING DEVICE

(71) Applicant: Jingsi XIE, SHENZHEN (CN)

(72) Inventors: Jingsi XIE, SHENZHEN (CN); Zhusong CHEN, SHENZHEN (CN); Jie LIN, SHENZHEN (CN); Hanhai ZHENG, SHENZHEN (CN)

(73) Assignee: Jingsi XIE, SHENZHEN (CN)

(21) Appl. No.: 18/804,166

(22) Filed: Aug. 14, 2024

Related U.S. Application Data
(63) Continuation-in-part of application No. PCT/CN2023/075840, filed on Feb. 14, 2023.

(30) Foreign Application Priority Data
Feb. 14, 2022 (CN) 202220284700.8
Aug. 12, 2022 (CN) 202222133982.4
Aug. 12, 2022 (CN) 202222134002.2
Aug. 24, 2022 (CN) 202222258693.7
Oct. 21, 2022 (CN) 202222777408.2
Oct. 21, 2022 (CN) 202222791476.4
Jan. 17, 2023 (CN) 202320169677.2

(51) Int. Cl. F04D 25/08; A61N 1/36; F04D 17/08; F04D 25/06; F04D 29/44

(52) U.S. Cl. CPC F04D 25/08; A61N 1/36014; F04D 17/08; F04D 25/0673; F04D 29/441

(57) ABSTRACT

A portable wind blowing device includes: a hanging body portion and a fan assembly. The hanging body portion defines at least one air inlet, a receiving cavity, and at least one air outlet. The fan assembly is received in the receiving cavity and defines at least one airflow intaking port corresponding to at least one air inlet. The air inlet is defined in at least one of: an inner side of the hanging body portion, an outer side of the hanging body portion, on an upper side of the hanging body portion, and a lower side of the hanging body portion. The fan assembly is configured to direct external air to flow through the air inlet into the hanging body portion and blow out the air through the air outlet.

### CROSS REFERENCE TO RELATED APPLICATIONS

The present application is a continuation-in-part application of the international patent application No. PCT/CN2023/075840, filed on Feb. 14, 2023. The international patent application No. PCT/CN2023/075840 claims the priority of: Chinese patent application No. 202220284700.8, filed on Feb. 14, 2022; Chinese patent application No. 202222133982.4, filed on Aug. 12, 2022; Chinese patent application No. 202222134002.2, filed on Aug. 12, 2022; Chinese patent application No. 202222258693.7, filed on Aug. 24, 2022; Chinese patent application No. 202222777408.2, filed on Oct. 21, 2022; Chinese patent application No. 202222791476.4, filed on Oct. 21, 2022; Chinese patent application No. 202320169677.2, filed on Jan. 17, 2023, and contents of which are incorporated herein by their entireties.

### TECHNICAL FIELD

The present disclosure relates to the field of fans, and in particular to a portable wind blowing device and a fan assembly thereof.

### BACKGROUND

As people's living standards improve, the users have increased demands on fans and desire the fans to have additional functions. However, fans in the art cannot be carried conveniently or have insufficient airflow. Therefore, the fans in the art cannot meet demands of the users.

A portable fan, such as a neck fan, is generally used by hanging on the neck of the user. The fan does not need to be held by hands. Therefore, hands of the user can be freed, and the portable fan can be used conveniently.

### SUMMARY OF THE DISCLOSURE

The present application disclose provides a portable wind blowing device, including: a hanging body portion and at least one fan assembly. The hanging body portion defines at least one air inlet, a receiving cavity, and at least one air outlet. At least one fan assembly is received in the receiving cavity. The fan assembly is arranged with: a centrifugal airflow zone; a mixed-flow booster blower, wherein the mixed-flow booster blower comprises diagonal fan blades, the diagonal fan blades are configured to direct the air from the exterior of the device into the centrifugal airflow zone and to generate a high pressure airflow; and an airflow concentration channel, configured to concentrate the high pressure airflow in the centrifugal airflow zone and to direct high pressure airflow to be blown out of the device through the at least one air outlet.

The present disclosure further provides a neck fan, including: a fan assembly and a body portion. The fan assembly includes a pressurizing centrifugal fan, wherein, the pressurizing centrifugal fan comprises fan blades, the fan blades are diagonal binary blade or ternary blades that are at least partially twisted. The body portion defines at least one air inlet and at least one air outlet and receiving the fan assembly. The body portion further defines one or more vent holes for air circulation, the fan assembly is configured to intake air through the at least one air inlet and blow the air out of the fan through the at least one air outlet, the one or more vent holes are configured to allow air located adjacent to a neck to flow freely. The pressurizing centrifugal fan is a mixed-flow pressurizing centrifugal fan, wherein, the mixed-flow pressurizing centrifugal fan comprises a motor for driving; the motor is a three-phase synchronous motor or a three-phase asynchronous motor and is configured to drive the mixed-flow pressurizing centrifugal fan to rotate at a predetermined rotation to generate a strong negative pressure to intake the air from an exterior of the neck fan.

The present disclosure further provides a centrifugal fan assembly, intaking air from a single side and configured in a portable wind blowing device. The centrifugal fan assembly includes a motor and fan blades, wherein the fan blades includes a hub and a plurality of airflow guiding blades. The hub includes a front side facing an air inlet of the portable wind blowing device and a rear side facing away from the air inlet of the portable wind blowing device; the plurality of the airflow guiding blades are arranged on and protrude from the front side; each of the plurality of airflow guiding blades comprises a blade top portion facing away from the front side. An airflow intaking space, through which the air is intaking from the single side, is formed between the front face of the hub and the blade top portion of the airflow guiding blade; and/or an axial vertical height between the blade top portion of the airflow guiding blade and the front side of the hub at least partially varies gradually in a radial direction of the hub.

### BRIEF DESCRIPTION OF THE DRAWINGS

FIG. 1 is an exploded perspective view of a portable wind blowing device according to an embodiment of the present disclosure.

FIG. 2 is a cross-sectional view of a fan assembly of the portable wind blowing device shown in FIG. 1, where a motor is omitted.

FIG. 3 is a structural schematic view of a mixed-flow booster blower of the fan assembly shown in FIG. 2, where a partial structure of an airflow concentration channel is shown by a dotted line.

FIG. 4 is a structural schematic view of an air blower of the fan assembly of the portable wind blowing device according to another embodiment of the present disclosure, where a partial structure of an airflow concentration channel is shown by a dotted line.

FIG. 5 is a perspective view of a neck fan according to an embodiment of the present disclosure.

FIG. 6 is an exploded view of a portion of the neck fan according to an embodiment of the present disclosure.

FIG. 7 is an exploded view of a portion of the neck fan according to an embodiment of the present disclosure.

FIG. 8 is a perspective view of the neck fan according to another embodiment of the present disclosure.

FIG. 9 is an exploded perspective view of the neck fan according to still another embodiment of the present disclosure.

FIG. 10 is an exploded perspective view of the neck fan according to still another embodiment of the present disclosure.

FIG. 11 is a cross-sectional view of the neck fan according to an embodiment of the present disclosure, where widths of the fan and the air duct are shown.

FIG. 12 is a perspective view of the neck fan according to another embodiment of the present disclosure.

FIG. 13 is a structural schematic view of an inner shell of the neck fan according to an embodiment of the present disclosure.

FIG. 14 is a structural schematic view of an outer shell of the neck fan according to an embodiment of the present disclosure.

FIG. 15 is a perspective view of the neck fan according to still another embodiment of the present disclosure.

FIG. 16 is an exploded view of a portion of the neck fan according to another embodiment of the present disclosure, where a decorative member is shown.

FIG. 17 is an exploded view of a portion of the neck fan according to another embodiment of the present disclosure.

FIG. 18 is an exploded view of a portion of the neck fan according to another embodiment of the present disclosure.

FIG. 19 is a perspective view of the neck fan according to another embodiment of the present disclosure.

FIG. 20 is an exploded view of a portion of the neck fan shown in FIG. 19, being viewed from a viewing angle.

FIG. 21 is an exploded view of a portion of the neck fan shown in FIG. 19, being viewed from another viewing angle.

FIG. 22 is an exploded view of a portion of the neck fan shown in FIG. 19, being viewed from still another viewing angle.

FIG. 23 is a structural schematic view of the neck fan according to another embodiment of the present disclosure, where a fan disposed inside the shell is taken out and shown.

FIG. 24 is a structural schematic view of the neck fan according to still another embodiment of the present disclosure, where a fan disposed inside the shell is taken out and shown.

FIG. 25 is an exploded view of a portion of the neck fan according to another embodiment of the present disclosure.

FIG. 26 is a perspective view of the neck fan according to another embodiment of the present disclosure, where a fan disposed inside the shell is taken out and shown.

FIG. 27 is a perspective view of the neck fan according to another embodiment of the present disclosure, where a fan disposed inside the shell is taken out and shown.

FIG. 28 is an exploded view of a portion of the neck fan according to still another embodiment of the present disclosure.

FIG. 29 is a schematic view of a portion of the neck fan shown in FIG. 28.

FIG. 30 is an exploded view of the neck fan shown in FIG. 28, being viewed from another viewing angle, where a control circuit board disposed on the other side is shown from a vent hole.

FIG. 31 is a perspective view of the neck fan according to another embodiment of the present disclosure, where a fan disposed inside the shell is taken out and shown.

FIG. 32 is a perspective view of the neck fan according to another embodiment of the present disclosure, where a fan disposed inside the shell is taken out and shown.

FIG. 33 is a perspective view of a portable wind blowing device configured with a fan assembly according to an embodiment of the present disclosure.

FIG. 34 is a cross-sectional view of the portable wind blowing device configured with the fan assembly according to an embodiment of the present disclosure.

FIG. 35 is an exploded view of a portion of the portable wind blowing device configured with the fan assembly according to an embodiment of the present disclosure.

FIG. 36 is a perspective view of blades of the fan assembly, being viewed from a viewing angle, according to an embodiment of the present disclosure.

FIG. 37 is a perspective view of blades of the fan assembly, being viewed from another viewing angle, according to an embodiment of the present disclosure.

FIG. 38 is a perspective view I of the portable wind blowing device according to an embodiment I of the present disclosure.

FIG. 39 is a perspective view II of the portable wind blowing device according to the embodiment I of the present disclosure.

FIG. 40 is a perspective view I of a portion of the portable wind blowing device according to the embodiment I of the present disclosure.

FIG. 41 is a perspective view II of the portion of the portable wind blowing device according to the embodiment I of the present disclosure.

FIG. 42 is a perspective view of an airflow concentration cover and an air guiding structure of the portable wind blowing device according to an embodiment I of the present disclosure.

FIG. 43 is a perspective view III of the portion of the portable wind blowing device according to the embodiment I of the present disclosure.

FIG. 44 is a perspective view III of the portable wind blowing device according to the embodiment I of the present disclosure.

FIG. 45 is a perspective view IV of the portion of the portable wind blowing device according to the embodiment I of the present disclosure.

FIG. 46 is a perspective view V of the portion of the portable wind blowing device according to the embodiment I of the present disclosure.

FIG. 47 is a perspective view of the portion of the portable wind blowing device according to an embodiment II of the present disclosure.

FIG. 48 is a perspective view of a fan assembly of the portable wind blowing device according to the embodiment II of the present disclosure.

FIG. 49 is a cross-sectional view of the fan assembly of the portable wind blowing device according to the embodiment II of the present disclosure.

### DETAILED DESCRIPTION

Technical solutions of the present disclosure is illustrated by specific embodiments. Any ordinary skilled person in the art shall understand other advantages and technical effects of the present disclosure based on the following disclosure.

In the following description, the accompanying drawings show various embodiments of the present disclosure. It is understood that other embodiments may be applied, and changes in mechanical composition, structures, electrics, and operations may be made without departing from the spirit and scope of the present disclosure. The following description shall not be considered as limiting the scope of the present disclosure. The scope of the present disclosure is is limited only by the claims. The terms used herein is used only to describe particular embodiments and is not intended to limit the present disclosure.

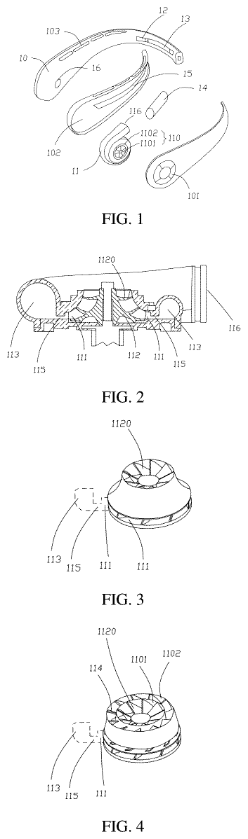

As shown in FIG. 1 to FIG. 3, FIG. 1 is an exploded perspective view of a portable wind blowing device according to an embodiment of the present disclosure, FIG. 2 is a cross-sectional view of a fan assembly of the portable wind blowing device shown in FIG. 1, and FIG. 3 is a structural schematic view of a mixed-flow booster blower of the fan assembly shown in FIG. 2.

In the present embodiments, the portable wind blowing device is provided and includes the following.

A hanging body 10 defines an air inlet 101, a receiving cavity 102, and an air outlet 103.

A fan assembly 11 is received in the receiving cavity 102.

The fan assembly 11 defines an air intaking port 110 corresponding to the air inlet 101. When the hanging body 10 is hanging around a neck of a user, the air inlet 101 is defined in an inner side of the hanging body 10 facing towards the neck; in an outer side of the hanging body 10 facing away from the neck; in an upper side of the hanging body 10 facing upwardly; and/or in a lower side of the hanging body 10 facing downwardly towards the ground. At least one of air intaking port 110 is defined in one side of the hanging body and is configured to direct external air to flow through the air inlet 101 to an interior of the hanging body 10 and to direct the air to flow out of the device through the air outlet 103.

To be noted that, in some embodiments, in case that a structure size is sufficient, for example, in a case that a size of a motor is getting smaller, a high rotation speed is achieved, and low power consumption is achieved, in the embodiments of the present disclosure, a plurality of fan assemblies 11 or a plurality of air inlets 101 may be arranged at a same location in order to ensure an air volume.

As shown in FIG. 2, according to a first embodiment of the portable wind blowing device of the present disclosure, the fan assembly 11 has following structure.

A centrifugal airflow zone 111 is formed.

A mixed-flow booster blower 112 includes diagonal blades 1120, and the diagonal blades 1120 are configured to direct the external air into the centrifugal airflow zone 111 and to generate a high pressure airflow.

An airflow concentration channel 113 is formed and configured to gather the high pressure airflow in the centrifugal airflow zone 111 and direct the high pressure airflow to flow out of the device from the air outlet 103.

To be noted that, in the present disclosure, a coaxial and double-sided mixed-flow pressurizing blower 112 may be arranged. That is, two mixed-flow pressurizing blowers 112, which are symmetrically disposed, may direct the external air simultaneously to flow, through two air inlets 101 defined in the inner side and the outer side of the hanging body, into the centrifugal airflow zone 111.

As shown in FIGS. 1 and 4, according to a second embodiment of the present disclosure, the fan assembly 11 is further arranged with an auxiliary blower 114. Two air inlets 110 are defined in the same side of the hanging body and includes the following.

A main air inlet 1101 is fluidly communicated with the mixed-flow booster blower 112 for forming the high pressure airflow.

An auxiliary air inlet 1102 is fluidly communicated with the auxiliary blower 114 for forming an auxiliary airflow.

The high pressure airflow and the auxiliary airflow are gathered in the airflow concentration channel 113.

As shown in FIG. 4, in a third embodiment according to the second embodiment, the auxiliary blower 114 sleeves a periphery of the mixed-flow booster blower 112. The auxiliary blower 114 may be a direct-flow centrifugal fan or a diagonal-flow centrifugal fan.

As shown in FIG. 2, FIG. 3 or FIG. 4, according to a fourth embodiment of the portable wind blowing device of the present disclosure, a compression limiting channel 115 is formed between the centrifugal airflow zone 111 and air concentration channel 113. A cross-sectional area of the compression limiting channel 115 is smaller than a cross-sectional area of the centrifugal airflow zone 111.

It is easily understood that, since the cross-sectional area of the compression limiting channel 115 is smaller than the cross-sectional area of the centrifugal airflow zone 111, it is ensured that the high pressure airflow pressurized for a secondary time when entering the airflow concentration channel 113. In addition, any airflow in the airflow concentration channel 113 is prevented from flowing reversely into the centrifugal airflow zone 111, ensuring the fan assembly 11 to continuously output the high-pressure airflow.

As shown in FIG. 1, according to a fifth embodiment of the portable wind blowing device of the present disclosure, the portable wind blowing device further includes following structure.

An electrical muscle stimulation (EMS) current pulse module 12 is configured to generate a microcurrent to act on the neck.

The EMS is also known as electroporation. The EMS microcurrent refers to stimulating and massaging facial muscles by transmitting, with a low level of electric current, mild and gentle electric waves to pass through the epidermis layer to reach the dermis layer of the face. In some embodiments, any EMS current pulse module available in the art may be configured in the device of the present embodiment and can be realized by a circuit that generates EMS currents.

In the present embodiment, due to the microcurrent generated by the EMS current pulse module 12, the cerebrum, the hypothalamus, the limbic reticular formation may be stimulated by currents having low intensity and specific waveforms, such that excitability of the brain can be regulated, insomnia and anxiety may be treated, or some symptoms may be relieved.

As shown in FIG. 1, according to a sixth embodiment of the portable wind blowing device of the present disclosure, the portable wind blowing device further includes the following structures.

A semiconductor refrigeration module 13 is arranged. Heat generated by the semiconductor refrigeration module 13 is dissipated to an exterior of the hanging body 10 via the high pressure airflow.

As shown in FIG. 1, according to a seventh embodiment of the portable wind blowing device of the present disclosure, the semiconductor refrigeration module 13 is integrally arranged with the EMS current pulse module 12.

To be noted that, since the semiconductor refrigeration module 13 and the EMS current pulse module 12 are integrally formed in the present embodiment, manufacturing costs can be are saved to a great extent. In addition, in a process of refrigeration and heating, weak electrical stimulation on the body based on the microcurrents and physiotherapy may be achieved, such that auxiliary functions of the device are enhanced.

In some embodiments, the hanging body 10 includes a flexible shell and a shape fixation member arranged inside the flexible shell, such that the hanging body 10 may wrap around an arm of the user, or the hanging body 10 may be twisted to change orientations of the air inlets 101.

In some embodiments, at least two fan assemblies 11 are arranged and are respectively disposed at two ends of the hanging body 10.

Alternatively, in some embodiments, at least one fan assembly 11 is arranged and is disposed at a middle of the hanging body 10. The airflow concentration channel 113 has at least two outputting ports 116 that output airflow towards the two ends of the hanging body 10, respectively.

In other embodiments, the portable wind blowing device of the present disclosure may further include a battery 14, an air guiding plate 15, a switch button 16, and a motor (not shown in the drawings), which are within an understandable scope of any ordinary skilled person in the art and will not be described herein.

In the present disclosure, the hanging body 10 allows the device to be easily carried by the user. When the hanging body 10 is hanging around the neck, the air inlets 101 are defined in the inner side of the hanging body 10 facing towards the neck, in the outer side of the hanging body 10 facing away from the neck, in the upper side of the hanging body 10 facing towardly, and/or in the lower side of the hanging body facing the ground. In addition, at least one of air intaking port 110 is defined on the same side, effectively enhancing an airflow volume.

### Embodiment I

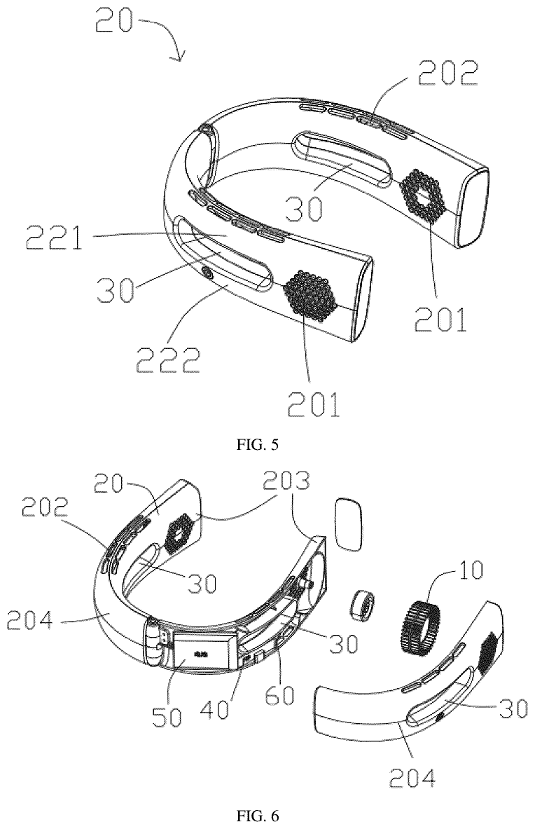

As shown in FIGS. 5 to 11, the present embodiment provides a neck fan that includes a fan 10 and a body portion 20. The body portion 20 is configured to define an air inlet 201, receive the fan 10, and define an air outlet 202. The body portion 20 further defines one or more vent holes 30 for air circulation. The fan 10 is configured to intake air through the air inlet 201 and to drive the air out of the neck fan from the air outlet 202. The vent holes 30 are defined to allow air adjacent to the neck to flow freely.

It is noted that the body portion 20 of the present embodiment includes an inner side 203 adjacent to the neck and an outer side 204 away from the neck. Each vent hole 30 extends through the inner side 203 and the outer side 204 to allow air at the outer side 204 to circulate with air located adjacent to the neck.

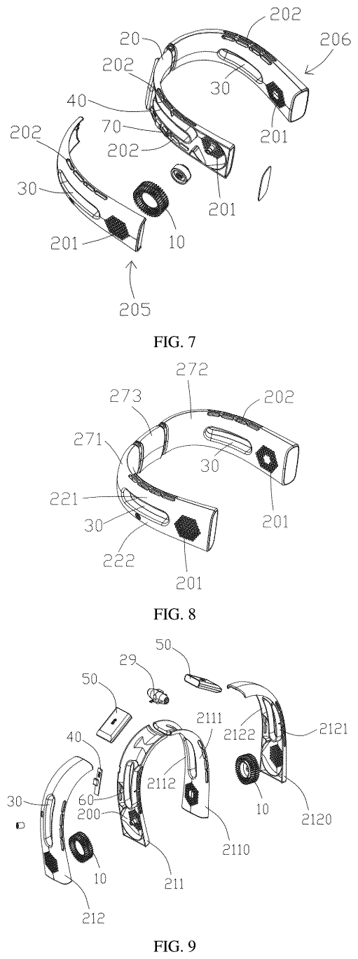

To be noted that, the one or more vent holes 30 may be one vent hole that extends along a length direction of the body portion 20. Alternatively, as shown in FIG. 7, two vent holes 30 may be defined and located at a first end 205 and a second end 206 of the body portion 20, respectively. Alternatively, as shown in FIG. 8, three vent holes 30 may be defined and respectively disposed at a first end 271, a second end 272, and a middle portion 273 of the body portion 20, where the middle portion 273 corresponds to a rear portion of the neck. Alternatively, as shown in FIG. 12, four or more vent holes 30 may be defined. The four or more vent holes 30 are distributed in an array along the length direction of the body portion 20 and are spaced apart from each other.

In the above embodiment in which one or more vent holes 30 are defined, an overall ventilation area of the vent holes 30 affects a direct ventilation and heat dissipation effect. For example, as the overall ventilation area increases, the heat dissipation effect is better. In the present embodiment, the overall ventilation area may be increased by increasing the number of the vent holes 30 or increasing an area of each vent hole 30. In practice, since the material of the body portion 20 affects structural strength of the device, the area of the vent holes 30 may be increased based on the material of the body portion 20, or a plurality of vent holes 30 may be defined in order to ensure the structural strength.

Specifically, as shown in FIG. 9, the body portion 20 includes an inner shell 211 and an outer shell 212. The inner shell 211 includes an inner shell body 2110, a first inner shell 2111 connected to the inner shell body 2110, and a second inner shell 2112 connected to the inner shell body 2110. The outer shell 212 includes an outer shell body 2120, a first outer shell 2121 connected to the outer shell body 2120, and a second outer shell 2121 connected to the outer shell body 2120. The inner shell body 2110 and the outer shell body 2120 are capped to the each other to form a receiving space 200. The first inner shell 2111 and the first outer shell 2121 are capped to the each other to form a first bridge portion 221. The second inner shell 2112 and the second outer shell 2122 are capped to the each other to form a second bridge portion 222. The first bridge portion 221 and the second bridge portion 222 are mated to each other to form the one or more vent holes 30.

To be added that, generally, a left portion of the body portion 20 and a right portion of the body portion 20 are substantially symmetrical to each other. Therefore, some reference numerals for drawings of the present disclosure are labeled at the first end 205, 271, and some other reference numerals are labeled at the second end 206, 272. Within the scope of that is understandable to any ordinary skilled person in the art, the reference numerals shall not be construed as a mislabeling. Of course, in other embodiments, the body portion 20 may have an asymmetrical structure. For example, only one fan assembly 10 is arranged and is disposed at only one end; or only one fan assembly is arranged and is disposed at the middle portion 273. Any ordinary skilled person in the art shall understand the technical solution in combination, which will not be described in detail.

The air inlet 201 of the present disclosure may be defined in the inner shell 211 and the outer shell 212. Alternatively, in an example where the neck fan is worn to the neck, the air inlets 201 are defined in the inner side 203, the outer side 204, a top surface and/or a bottom surface of the body portion 20. Locations of the air inlets 201 are determined according to demands of a ventilation volume or a noise level, which will not be limited herein. In addition, the air outlets 202 of the present disclosure may be defined in the inner side 203, the top surface and/or the bottom surface according to the actual demands. Locations of the air outlets 202 may be determined according to demands of airflow blowing positions and the noise level, which will not be limited herein.

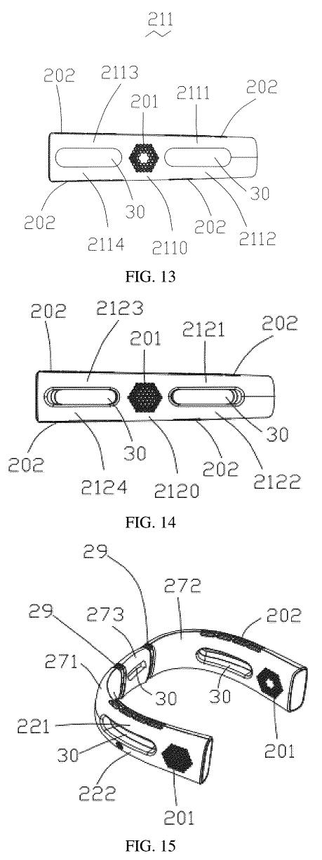

To be added that, a cross section of a ventilation channel of the vent hole 30 of the present embodiment may be elongated-circular (as shown in FIGS. 13 and 14), circular, wavy, or in a shape similar to an overall shape of the body portion 20. A plurality of circular vent holes 30 may be arranged. The wavy vent hole 30 may extend along the length direction of the body portion 20. The vent hole 30 having the shape similar to the overall shape of the body portion 20 enables the entire product to have a consistent visual effect and enables the entire product to be light and thin.

In the present disclosure, a fan blade impeller having a conventional size or a larger radius may be arranged to ensure an air blowing effect and the heat dissipation effect. While an overall size of the body portion 20 is increased, the vent holes 30 are configured properly to form ventilation and to achieve heat dissipation for the neck. In this way, a steamer-type closed space, which may be formed due to the large-sized body portion, can be avoided. By defining the venti holes 30 and a plurality of variable air ducts inside the body portion 20, redundancy of an internal structure of the body portion 20 is prevented. In this way, all functional components are located at suitable positions without filling inside the body portion 20 as what is designed in the art. The vent holes 30 are defined to form variable air ducts, such that heat-generating components can be isolated from other components, and in particular, highly heat components, such as a control circuit board may be indepdently located in any side wall of the vent hole 30. Since the vent holes 30 are defined in the body portion 20, a contact surface area between the body portion 20 and the external air is directly increased. In addition, since the air is flowing smoothly, heat emitted from components inside the neck fan are quickly dissipated through the increased contact surface area, preventing the heat from accumulating inside the body portion 20. By defining the vent holes 30, less material is used to produce the neck fan of the present disclosure, such that the neck fan is thinner and lighter. The neck fan is prevented from being excessively heavy and applying unnecessary weight to the neck of the user.

The inner shell 211 and the outer shell 212 are not limited to being capped with each other from an inner side to an outer side, but the inner shell 211 and the outer shell 212 may be formed as a one-piece and integral structure. Alternatively, the shell is formed by a bottom cover and a top cover that can be disassembled from each other, which is not limited herein.

In some embodiments, as shown in FIG. 6, the neck fan further includes a control circuit board 40, a battery 50, and wires, which will be described below by referring to different configuration applications.

In an application example 1, as shown in FIG. 7, the receiving space 200 is defined to receive the fan assembly 10. A first air duct is formed in the first bridge portion 221, and the air outlets 202 are defined in an inner side, an upper side and/or a lower side of the first bridge portion 221. The second bridge portion 222 defines a second air duct, and an inner and/or an upper side of the second bridge portion 221 defines the air outlets 202. In this way, the first bridge portion 221 is configured to blow the airflow towards and cool the face. By defining the air outlets 202 at locations where the vent holes 30 are defined, air ventilation at the vent holes 30 is further improved.

In an application example 2, not shown in the drawings, the receiving space 200 is defined to receive the fan assembly 10. The first air duct is formed in the first bridge portion 221, and the air outlets 202 are formed in the inner side, the upper side and/or the lower side of the first bridge portion 221. The control circuit board 40, the battery 50, and the wires electrically connected to the battery 50 are arranged inside the second bridge portion 222. Since additional vent holes 30 are defined in the body portion 20, the contact surface area between the body portion 20 and the external air is directly increased. In addition, since the air is flowing smoothly, heat emitted by the control circuit board 40 and the battery 50 inside the neck fan can be quickly dissipated out of the neck fan through the increased contact surface area, preventing the heat from accumulating inside the body portion 20.

In an application example 3, not shown in the drawings, the receiving space 200 is defined to receive the fan assembly 10, the control circuit board 40, the battery 50, and the wires electrically connected to the battery 50. The first air duct is formed in the first bridge portion 221, and air outlets 202 are defined in the inner side, the upper side, and/or the lower side of the first bridge portion 221.

In an application example 4, not shown in the drawings, one or more fan assemblies 10 are mounted inside the first bridge portion 221 and/or the second bridge portion 222. The battery 50, the control circuit board 40, and the wires electrically connected to the battery 50 are received in the receiving space 200. In the present application example, a plurality of small-sized fan assemblies may be arranged to dissipate the heat, ensuring a more uniform blowing effect and preventing the airflow from being blown out of the neck fan in a concentrated manner.

In an application example 5, as shown in FIGS. 6 and 9, the fan assembly 10 is mounted in the receiving space 200. An end of the first bridge portion 221 away from the receiving space 200 is communicated to an end of the second bridge portion 222 away from the receiving space 200. The semiconductor refrigeration module 60 is mounted in the second bridge portion 222. The fan assembly 10 directs the air, which is intaken from the air inlet 201, to the inside of the second bridge portion 222 to be cooled/heated by the semiconductor refrigeration module 60. The fan assembly 10 further directs the cooled/heated air to flow into the air duct in the first bridge portion 221 via the communicated end and directs the cooled/heated air to flow out of the neck fan through the air outlets 202 of the first bridge portion 221. In the present application example, the air duct provides a longer flowing path and time to cool/heat the air. In this way, a poor cooling effect caused by a low power conversion of the semiconductor refrigeration module 60 is prevented, ensuring that the airflow blown out of the neck fan meets temperature demands.

In an application example 6, the fan assembly 10 is mounted in the receiving space 200. The first air duct is formed in the first bridge portion 221. The air outlets 202 are defined in the inner side and/or the upper side of the first bridge portion 221. A negative-ion generating member 70 is mounted in the second bridge portion 222, as shown in FIG. 7. In the present application example, negative ions released by the negative-ion generating member 70 reach the neck after flowing out of the vent holes 30 and are prevented from being blown away by the airflow of the neck fan, ensuring a usage effect and a concentration of the negative ions.

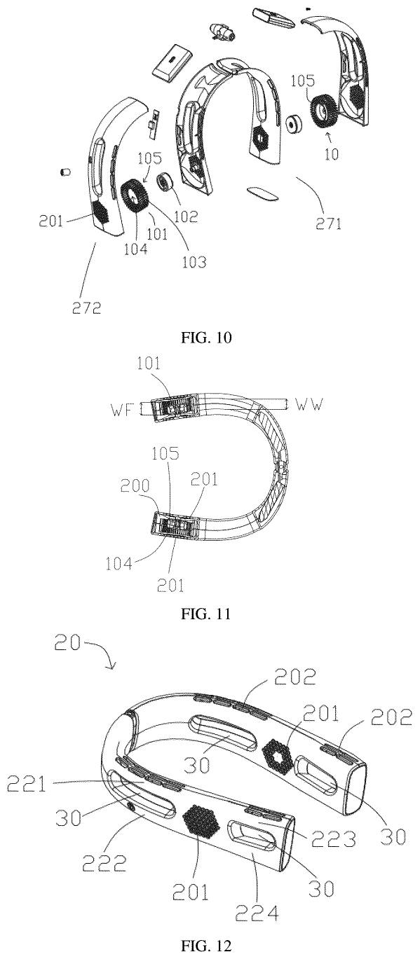

In the present embodiment, as shown in FIG. 10, the fan assembly 10 of the present disclosure includes a centrifugal fan 101. The centrifugal fan 101 includes a motor 102, fan blades 103, a first air intaking region 104, and a second air intaking region 105. The first air intaking region 104 and the second air intaking region 105 are formed respectively at two end surfaces of the fan blades 103 and are far away from each other. The body portion 20 defines the air inlets 201 that are communicated with the first air intaking region 104 and the second air intaking region 105.

Further, as shown in FIG. 11, the fan assembly 10 includes the centrifugal fan 101, and the centrifugal fan 101 includes the motor 102, the fan blades 103, the first air intaking region 104, and the second air intaking region 105. The first air intaking region 104 and the second air intaking region 105 are formed respectively at two end surfaces of the fan blades 103 and are far away from each other. In a direction where the outer side 204 and the inner side 203 are connected to each other, a maximum width WW of the air ducts formed in the corresponding bridge portions of the body portion 20 is less than or equal to a fan blade thickness WF, which is a distance between the two end faces of the fan blade 103. In some embodiments, the maximum width WW is less than the fan blade thickness WF to pressurize the airflow entering the bridge portion in a width of the air duct.

To be noted that, as shown in FIG. 6, in the present embodiment, the battery 50 is disposed on a side of the vent hole 30 away from the fan assembly 10, and the control circuit board 40 is disposed in the second bridge portion 222 and between the battery 50 and the fan assembly 10. In this way, the fan assembly 10, the control circuit board 40, and the battery 50 are spaced apar from each other and disposed in a circumferential direction around the vent hole 30. Therefore, heat of a circulation airflow of the body portion 20 can be dissipated through the vent hole 30.

### Embodiment II

As shown in FIGS. 5 to 11 and FIGS. 12 to 14, in the present embodiment, the body portion 20 may include the inner shell 211 and the outer shell 212. The inner shell 211 includes the inner shell body 2110, the first inner shell 2111 connected to the inner shell body 2110, and the second inner shell 2112 connected to the inner shell body 2110. The outer shell 212 includes the outer shell body 2120, the first shell connected to the outer shell body 2120, and the second shell connected to the outer shell body 2120. The inner shell body 2110 and the outer shell body 2120 are capped to each other to form the receiving space 200. The first inner shell 2111 and the first outer shell 2121 are capped to each other to form the first bridge portion 221. The second inner shell 2112 and the second outer shell 2122 are capped to each other to form the second bridge portion 222. The first bridge portion 221 and the second bridge portion 222 are mated to each other to form the one or more vent holes 30.

In the present embodiment, the inner shell 211 further includes a third inner shell 2113 connected to the inner shell body 2110 and a fourth inner shell 2114 connected to the inner shell body 2110. The outer shell 212 further includes a third outer shell 2123 connected to the outer shell body 2120 and a fourth outer shell 2124 connected to the outer shell body 2120. The third inner shell 2113 and the third outer shell 2123 are capped to each other to form a third bridge portion 223. The fourth inner shell 2114 and the fourth outer shell 2124 are capped to each other to form a fourth bridge portion 224. The first bridge portion 221 and the third bridge portion 223 are disposed at different sides of the receiving space 200. The second bridge portion 222 and the fourth bridge portion 224 are disposed at different sides of the receiving space 200.

Accordingly, in the present embodiment, various application examples may be applied depending on the number of bridge portions.

In an application example 7, the fan assembly 10 is mounted in the receiving space 200. Each of the first bridge portion 221, the second bridge portion 222, the third bridge portion 223 and the fourth bridge portion 224 defines a respective air duct therein and defines respective air outlets 202 in the inner side and/or the upper side thereof.

In an application example 8, the fan assembly 10 is mounted in the receiving space 200. Each of the first bridge portion 221 and the third bridge portion 223 defines the respective air duct therein and defines respective air outlets 202 in the inner side and/or the upper side thereof. The battery 50 and/or the control circuit board 40 are mounted in the second bridge portion 222 and/or the fourth bridge portion 224.

To be noted that, in the present disclosure, the battery 50 mounted in the bridge portion may be a lithium battery having a shape matching with a shape of the bridge portion, such as a bar-shaped battery in a model 18650. The battery 50 mounted in the receiving space 200 may be a polyhedral battery adapted to the receiving space, such as a polymer battery.

In an application example 9, the fan assembly 10 is mounted in the receiving space 200. Each of the first bridge portion 221 and the third bridge portion 223 defines a respective air duct and defines respective air outlets 202 on the inner side and/or the upper side thereof. The end of the first bridge portion 221 away from the receiving space 200 is communicated with the end of the second bridge portion 222 away from the receiving space 200. The end of the third bridge portion 223 away from the receiving space 200 is communicated with the end of the fourth bridge portion 224 away from the receiving space 200. The semiconductor refrigeration module 60 is mounted in the second bridge portion 222. The fan assembly 10 directs the airflow, which is intaken from the air inlets 201, into the second bridge portion 222 to be cooled/heated by the semiconductor refrigeration module 60. The fan assembly 10 then directs the cooled/heated airflow into the air duct in the first bridge portion 221 through the communicated end and drives the cooled/heated airflow out of the neck fan through the air outlets 202 defined in the first bridge portion 221. The semiconductor refrigeration module 60 is mounted in the fourth bridge portion 224. The fan assembly 10 directs the airflow, which is intaken from the air inlets 201, into the fourth bridge portion 224 to be cooled/heated by the semiconductor refrigeration module 60. The fan assembly 10 then directs the cooled/heated airflow into the air duct in the third bridge portion 223 through the communicated end and drives the cooled/heated airflow out of the neck fan through the air outlets 202 defined in the third bridge portion 223.

In an application example 10, the fan assembly 10 is mounted in the receiving space 200. The first bridge portion 221, the second bridge portion 222, the third bridge portion 223, and the fourth bridge portion 224 are fluidly communicated to each other to form a cooling circulation. The semiconductor refrigeration module 60 is mounted inside each of at least three of the first bridge portion 221, the second bridge portion 222, the third bridge portion 223, and the fourth bridge portion 224. The fan assembly 10 directs the airflow, which is intaken from the air inlets 201, into the cooling circulation to be cooled/heated by the semiconductor refrigeration modules 60 and directs the cooled/heated airflow to be blown out of the neck fan through the air outlets 202. The air outlets 202 are defined in a last bridge portion of the cooling circulation and/or located on an air outlet duct that is located at an upper portion of the receiving space 200 and is spaced apart from the receiving space 200.

In application example 11, the fan assembly 10 is mounted in the receiving space 200. Each of the first bridge portion 221 and the third bridge portion 223 defines the respective air duct therein and defines the respective air outlets 202 in the inner side and/or the upper side thereof. The negative-ion generating member 70 is arranged inside each of the second bridge portion 222 and/or the fourth bridge portion 224.

In the application example 9 and the application example 10, a plurality of bridge portions are mated to each other to form a relatively long air duct, such that a cooling effect caused by the semiconductor refrigeration module 60 is improved, and the cooled airflow is blown out of the neck fan through the air outlets 202.

To be noted that, in the above embodiments, the air outlets 202 are not limited to or fixed at positions as what is shown in one or more drawings. Instead, the air outlets 202 may be defined or closed according to the demands of various application examples, which can be understood according to the application examples.

In an embodiment 3, as shown in FIG. 7, the body portion 20 of the present embodiment includes a first neck portion 205 and a second neck portion 206 which respectively hang at two sides of the neck of the user. The first neck portion 205 and the second neck portion 206 are hinged to each other. The vent holes 30 are defined in the first neck portion 205 and the second neck portion 206. Specifically, as shown in FIG. 9, a hinge mechanism 29 is disposed between the first neck portion 205 and the second neck portion 206 to hinge the first neck portion 205 with the second neck portion 206.

In an embodiment 4, as shown in FIG. 15, the body portion 20 includes a first neck portion 271, a second neck portion 272, and a middle portion 273. The middle portion 273 is disposed corresponding to the rear of the neck. The first neck portion 271 and the second neck portion 272 hang respectively at two sides of the neck of the user. Each of the first neck portion 271 and the second neck portion 272 takes a respective hinge mechanism 29 to hinge with the middle portion 273. The vent holes 30 are defined in the first neck portion 271, the second neck portion 272, and/or the middle portion 273. In the present embodiment, the hinge mechanism 29 may be a universal ball, a hinge, or the like, which is not limited herein.

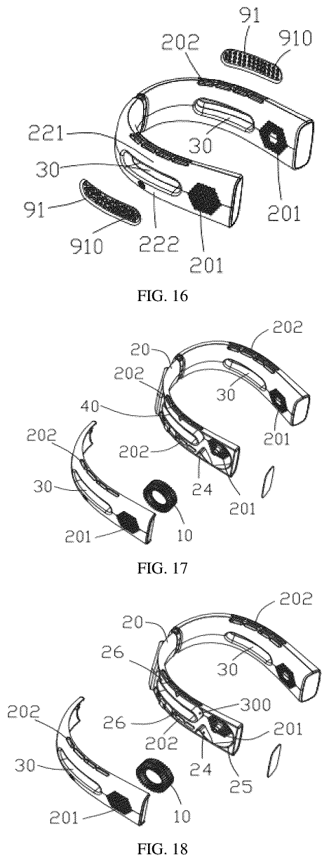

In an embodiment 5, as shown in FIG. 16, the neck fan may further include a decorative member 91. The decorative member 91 is disposed corresponding to each vent hole 30. The decorative member 91 is a cover plate having mesh holes 910 and is used to cover the vent hole 30. Alternatively, the decorative member 91 is hinged to the body portion 20 to regulate the ventilation area of the exposed vent hole 30.

Of course, when the decorative member 91 is hinged with the body portion 20, and when the semiconductor refrigeration module 60 is arranged and the neck fan is being used in winter, the decorative member 91 may completely cover and close the vent hole 30. In this way, it is ensured that the airflow heated by the semiconductor refrigeration module 60 reserves heat to the neck and the face, preventing the external cold air from blowing towards the neck from the vent holes 30, ensuring the usage effect of the neck fan.

In an embodiment 6, as shown in FIG. 17, the neck fan may further include an imitative vortex tongue structure 24. The imitative vortex tongue structure 2424 is disposed to at least partially surround the fan assembly 10. The imitative vortex tongue structure 2424 is formed inside the receiving space 200.

In other embodiments, the imitative vortex tongue structure 24 may be integrally formed with one or more of bridge portions to be multiplexed used. Specifically, as shown in FIG. 18, a bridge cover plate 300 is formed on the inner shell 211 surrounding the vent holes 30. The imitative vortex tongue structure 24 and the bridge cover plate 300 may be integrally formed with each other to be multiplexed used, a reduced number of plate members are used.

In some embodiments, the neck fan may further include an imitative vortex shell structure 25. The imitative vortex shell 25 may be disposed in an Archimedean helix manner to surround the fan assembly 10. In other embodiments, the imitative vortex shell 25 may be integrally formed with the inner shell 211 and/or the outer shell 212. For example, a structure of the inner shell 211 and/or the outer shell 212 that is configured to arrange the vortex shell of the fan assembly 10 may be changed, such that the structure has a shape consistent with an overall shape of the imitative vortex shell 25. In this way, an independent imitative vortex shell 25 may not be needed. The neck fan may have a more simple structure and a reduced weight.

As shown in FIG. 18, the neck fan may further include an air-duct partition plate 26. The air-duct partition plate 26 is formed in the respective air duct of each bridge portion, such that an airflow volume or an airflow pressure can be adjusted according to a distance between the air outlets 202 and the fan assembly 10.

In the present embodiment, a cross-sectional area of the air duct formed by the air-duct partition plate 26 is gradually reduced along a direction that the airflow is blown out of the neck fan. In this way, a difference between airflow pressures at various air outlets is controlled within a preset range, such that the airflow pressure at the air outlet 202, which is located away from the fan assembly 10, is prevented from being excessively small.

In an embodiment 7, as shown in FIGS. 5 to 11, the present disclosure further provides an air duct apparatus. The air duct apparatus is configured in the neck fan of any of the above embodiments. The air duct apparatus includes a body portion 20. The body portion 20 defines the air inlets 201 and the air outlets 202. The body portion 20 defines one or more vent holes 30 for air circulation.

The body portion 20 includes the inner side 203 adjacent to the neck and the outer side 204 away from the neck. Each vent hole 30 extends through the inner side 203 and the outer side 204 to allow air at the outer side 204 to be circulated with air located adjacent to the neck. One vent hole 30 may be defined and extends along the length direction of the body portion 20. Alternatively, two vent holes 30 are defined and are located at the first end 205 and the second end 206 the body portion 20. Alternatively, three vent holes 30 are arranged and are respectively disposed at the first end 205, the second end 206, and the middle portion of the body portion 20, where the middle portion corresponds to the rear of the neck. Alternatively, four or more vent holes 30 are defined. The four or more vent holes are arranged along the length direction of the body portion 20 into an array and are disposed spaced apart from each other.

In the present embodiment, the body portion 20 includes the inner shell 211 and the outer shell 212. The inner shell 211 includes the inner shell body 2110, the first inner shell 2111 connected to the inner shell body 2110, and the second inner shell 2112 connected to the inner shell body 2110. The outer shell 212 includes the outer shell body 2120, the first outer shell 2121 connected to the outer shell body 2120, and the second outer shell 2122 connected to the outer shell body 2120. The inner shell body 2110 and the outer shell body 2120 are capped to each other to form the receiving space 200. The first inner shell 2111 and the first outer shell 2121 are capped to each other to form the first bridge portion 221. The second inner shell 2112 and the second outer shell 2122 are capped to each other to form the second bridge portion 222. The first bridge portion 221 and the second bridge portion 222 are mated to each other to form the one or more vent holes 30.

In the present embodiment, a plurality of application examples are as follows.

The fan assembly 10 is mounted in the receiving space 200. The first air duct is formed in the first bridge portion 221. Each of the inner side, the upper side and/or the lower side of the first bridge portion 221 defines the air outlets 202. The second air duct is defined in the second bridge portion 222. Each of the inner side and/or the upper side of the second bridge portion 222 defines the air outlets 202.

Alternatively, the fan assembly 10 is mounted in the receiving space 200. The first air duct is formed in the first bridge portion 221. Each of the inner side, the upper side and/or the lower side of the first bridge portion 221 defines the air outlets 202. The control circuit board 40, the battery 50, and the wires electrically connected to the battery 50 are mounted inside the second bridge portion 222.

Alternatively, the fan assembly 10, the control circuit board 40, the battery 50, and the wires electrically connected to the battery 50 are mounted in the receiving space 200. The first air duct is formed in the first bridge portion 221. Each of the inner side, the upper side and/or the lower side of the first bridge portion 221 defines the air outlets 202.

Alternatively, one or more fan assemblies 10 are mounted in the first bridge portion 221 and/or the second bridge portion 222. The control circuit board 40, the battery 50, and the wires electrically connected to the battery 50 are mounted in the receiving space 200.

Alternatively, the fan assembly 10 is mounted in the receiving space 200. The end of the first bridge portion 221 away from the receiving space 200 and the end of the second bridge portion 222 away from the receiving space 200 are communicated with each other. The semiconductor refrigeration module 60 is mounted inside the second bridge portion 222. The fan assembly 10 directs the air, which is intaken from the air inlets 201, into the second bridge portion 222 to be cooled/heated by the semiconductor refrigeration module 60. The fan assembly 10 then directs the cooled/heated air into the first bridge portion 221 through the communicated end and directs the cooled/heated air to be blown out of the neck fan through the air outlets 202 in the first bridge portion 221.

Alternatively, the fan assembly 10 is mounted in the receiving space 200. The first air duct is defined in the first bridge portion 221, and each of the inner side and/or the upper side of the first bridge portion 221 defines the air outlets 202. The negative-ion generating member 70 is mounted inside the second bridge portion 222.

In an eighth embodiment, as shown in FIGS. 5 to 11 and FIGS. 12 to 14, the present disclosure further provides an air duct apparatus configured in the neck fan of any of the above embodiments. The air duct apparatus includes the body portion 20. The body portion 20 includes the inner shell 211 and the outer shell 212. The inner shell 211 includes the inner shell body 2110, first inner shell 2111 connected to the inner shell body 2110, the second inner shell 2112 connected to the inner shell body 2110, the third second inner shell 2112 connected to the inner shell body 2110, and the fourth inner shell 2114 connected to the inner shell body 2110. The outer shell 212 includes the outer shell body 2120, the first outer shell 2121 connected to the outer shell body 2120, the second outer shell 2122 connected to the outer shell body 2120, the third outer shell 2123 connected to the outer shell body 2120, and the fourth outer shell 2124 connected to the outer shell body 2120. The inner shell body 2110 and the outer shell body 2120 are capped to each other to form the receiving space 200. The first inner shell 2111 and the first outer shell 2121 are capped to each other to form the first bridge portion 221. The second inner shell 2112 and the second outer shell 2122 are capped to each other to form the second bridge portion 222. The first bridge portion 221 and the second bridge portion 222 are mated to each other to define the one or more vent holes 30. The third inner shell 2113 and the third outer shell 2123 are capped to each other to form the third bridge portion 223. The fourth inner shell 2114 and the fourth outer shell 2124 are capped to each other to form the fourth bridge portion 224. The first bridge portion 221 and the third bridge portion 223 are located at different sides of the receiving space 200, and the second bridge portion 222 and the fourth bridge portion 224 are located at different sides of the receiving space 200.

The air duct apparatus of the present embodiment may include a plurality of application examples as follows.

In the present embodiment, the fan assembly 10 is mounted in the receiving space 200. Each of the first bridge portion 221, the second bridge portion 222, the third bridge portion 223, and the fourth bridge portion 224 defines the respective air duct therein and defines the respective air outlets 202 in the inner side and/or the upper side thereof.

Alternatively, the fan assembly 10 is mounted in the receiving space 200. Each of the first bridge portion 221 and the third bridge portion 223 defines the respective air duct therein and defines the respective air outlets 202 in the inner side and/or the upper side thereof. The battery 50 and/or the control circuit board 40 are mounted in the second bridge portion 222 and/or the fourth bridge portion 224.

Alternatively, the fan assembly 10 is mounted in the receiving space 200. Each of the first bridge portion 221 and the third bridge portion 223 defines the respective air duct therein and defines the respective air outlets 202 in the inner side and/or the upper side thereof. The end of the first bridge portion 221 away from the receiving space 200 is communicated with the end of the second bridge portion 222 away from the receiving space 200. The end of the third bridge portion 223 away from the receiving space 200 is communicated with the end of the fourth bridge portion 224 away from the receiving space 200. The semiconductor refrigeration module 60 is mounted in the second bridge portion 222. The fan assembly 10 directs the air, which is intaken from the air inlets 201, into the second bridge portion 222 to be cooled/heated by the semiconductor refrigeration module 60. The fan assembly 10 then directs the cooled/heated air into the first bridge portion 221 through the communicated end and directs the cooled/heated air to be blown out of the neck fan through the air outlets 202 in the first bridge portion 221. The semiconductor refrigeration module 60 is mounted in the fourth bridge portion 224. The fan assembly 10 directs the air, which is intaken from the air inlets 201, into the fourth bridge portion 224 to be cooled/heated by the semiconductor refrigeration module 60. The fan assembly 10 then directs the cooled/heated air into the third bridge portion 223 through the communicated end and directs the cooled/heated air to be blown out of the neck fan through the air outlets 202 in the third bridge portion 223.

Alternatively, the fan assembly 10 is mounted in the receiving space 200. The first bridge portion 221, the second bridge portion 222, the third bridge portion 223, and the fourth bridge portion 224 are fluidly communicated to each other to form the cooling circulation. The semiconductor refrigeration module 60 is arranged in each of at least three of the first bridge portion 221, the second bridge portion 222, the third bridge portion 223, and the fourth bridge portion 224. The fan assembly 10 directs the air, which is intaken from the air inlets 201, into the cooling circulation to be cooled/heated by the semiconductor refrigeration module 60 and then directs the cooled/heated air to be blown out of the neck fan through the air outlets 202. The air outlets 202 are located in the last bridge portion of the cooling circulation and/or on the air outlet duct, which is located at the upper portion the receiving space 200 and is spaced apart from the receiving space 200.

The fan assembly 10 is mounted in the receiving space 200. Each of the first bridge portion 221 and the third bridge portion 223 defines the respective air duct therein and defines the respective air outlets 202 in the inner side and/or the upper side thereof. The negative-ion generating member 70 is mounted in the second bridge portion 222 and/or the fourth bridge portion 224.

As shown in FIG. 7, the body portion 20 of the air duct apparatus of the present embodiment includes the first neck portion 205 and the second neck portion 206 that respectively hang on two sides of the neck of the user. The first neck portion 205 and the second neck portion 206 are hinged to each other. The vent holes 30 are defined in the first neck portion 205 and the second neck portion 206. Specifically, as shown in FIG. 9, the first neck portion 205 and the second neck portion 206 are hinged to each other by the hinge mechanism 29.

As shown in FIG. 15, the body portion 20 of the air duct apparatus includes the first neck portion 271, the second neck portion 272, and the middle portion 273, where the middle portion 273 is located corresponds to the rear of the neck. The first neck portion 205 and the second neck portion 206 respectively hang on two sides of the neck of the user. Each of the first neck portion 205 and the second neck portion 206. is hinged to the middle portion 273 by the respective hinge mechanism 29. The vent holes 30 are defined in the first neck portion 271, the second neck portion 272, and/or the middle portion 273. In the present embodiment, the hinge mechanism 29 may be a universal ball, a hinge, and so on, which s not limited herein.

As shown in FIG. 16, the air duct apparatus further includes the decorative member 91, the decorative member 91 is disposed corresponding to each vent hole 30. The decorative member 91 is a cover plate having mesh holes 910 and is configured to cover the corresponding vent hole 30. Alternatively, the decorative member 91 is hinged to the body portion 20 to regulate the ventilation area of the vent hole 30.

Of course, when the decorative member 91 is hinged to the body portion 20, and when the semiconductor refrigeration module 60 is arranged and used in winter, the vent holes 30 can be completely covered. It is ensured that the airflow heated by the semiconductor refrigeration module 60 reserves heat to the neck and the face, preventing the cold air out of the neck fan from flowing, through the vent holes 30, to reach the neck, and ensuring the usage effect.

As shown in FIG. 17, the air duct apparatus may further include the imitative vortex tongue structure 24. The imitative vortex tongue structure 24 at least partially surround the fan assembly 10. The imitative vortex tongue structure 24 is formed in the receiving space 200.

In other embodiments, the imitative vortex tongue structure 24 may be integrally formed with the one or more of the bridge portions to be multiplexed used. Specifically, as shown in FIG. 18, a bridge cover plate 300 is formed on the inner shell 211 and surrounds the vent holes 30. The imitative vortex tongue structure 24 and the bridge cover plate 300 may be integrally formed with each other to multiplexed used, such that a reduced number of plates may be arranged.

In some embodiments, the air duct apparatus may be arranged with an imitative vortex shell 25. The imitative vortex shell 25 may be disposed in an Archimedean helix manner to surround the fan assembly 10. In other embodiments, the imitative vortex shell 25 may be integrally formed with the inner shell 211 and/or the outer shell 212. For example, a structure of the inner shell 211 and/or the outer shell 212 that is configured to arrange the vortex shell of the fan assembly 10 may be changed, such that the structure has a shape consistent with an overall shape of the imitative vortex shell 25. In this way, an independent imitative vortex shell 25 may not be needed. The neck fan may have a more simple structure and a reduced weight.

As shown in FIG. 18, the air duct apparatus may further include an air-duct partition plate 26. The air-duct partition plate 26 is formed in the respective air duct of each bridge portion, such that an airflow volume or an airflow pressure can be adjusted according to a distance between the air outlets 202 and the fan assembly 10.

In the present embodiment, a cross-sectional area of the air duct formed by the air-duct partition plate 26 is gradually reduced along a direction that the airflow is blown out of the neck fan.

In this way, a difference between airflow pressures at various air outlets is controlled within a preset range, such that the airflow pressure at the air outlet 202, which is located away from the fan assembly 10, is prevented from being excessively small.

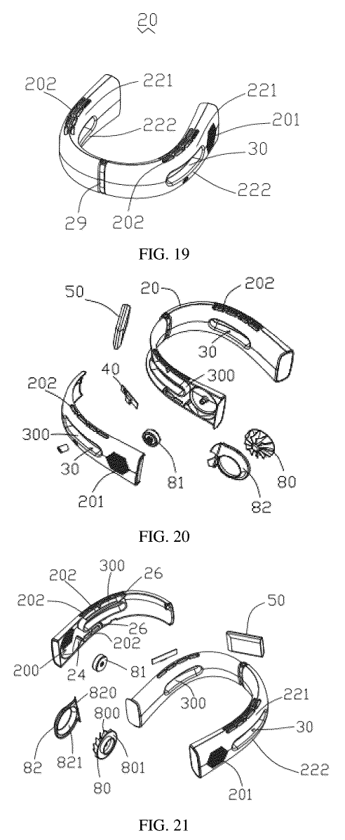

In an embodiment 9, as shown in FIGS. 19 to 22, the neck fan may include the fan assembly and the body portion 20. The body portion 20 defines the air inlets 201 and the air outlets 202 and is arranged with the fan assembly. The body portion 20 define one or more vent holes 30 for air flowing. The fan assembly is configured to intake air through the air inlets 201 and drives the air to be blown out of the neck fan through the air outlets 202. The vent holes 30 are configured to allow air adjacent to the neck to flow freely. The body portion 20 includes the inner side 203 adjacent to the neck and the outer side 204 away from the neck. Each vent hole 30 extends through the inner side 203 and the outer side 204 to allow the air at the outer side 204 to circulate with the air adjacent to the neck.

In the present embodiment, the fan assembly includes a pressurizing centrifugal fan. The pressurizing centrifugal fan includes a motor 81 and fan blades 80. The fan blades 80 are ternary blades, and at least portion of the ternary blades are twisted. The twisted ternary blades has concave portions and convex portions and have a flower-petal shape in overall. The twisted ternary blades is not arranged on a same plane or a same curved surface. The twisted configuration of the ternary blades increases the airflow volume and the airflow pressure inside the neck fan. The ternary blades ensure that airflow speeds at the air outlets 202 are uniform with each other, the airflow pressure is distributed properly, and a loss of the airflow is reduced.

Specifically, the fan blades 80 includes a mounting base plate 801 and the ternary blades 800 arranged on the mounting base plate 801. The ternary blades 800 may be fixed to the mounting base plate 801 by snapping or ultrasonics, or the ternary blades 800 and the mounting base plate 801 are integrally formed as a one-piece structure. In some embodiments, the ternary blades 800 and the mounting base plate 801 may be made of metal, if the cost permits, to enhance a pass rate of finished products. A side of the mounting base plate 801 away from the ternary blades 800 defines a mounting hole for mounting the motor 81.

In some embodiments, a wind guiding cone may be arranged on the other side of the mounting base plate 801 where the ternary blades 800 are arranged. In this way, the airflow pressure is further improved, and an air delivery distance is increased.

Further, the pressurizing centrifugal fan further includes an air guiding cover 82. The air guiding cover 82 is disposed opposite to the fan blades 80 and corresponding to the air inlets 201. The air guiding cover 8282 includes a cover plate 820 and a curved cover body 821. The curved cover body 821 is formed by extending from the cover plate 820. An inner surface of the curved cover body 821 corresponding to the ternary blades 800 is adapted with an outer contour of the ternary blades 800, such that the airflow pressure is increased. In some embodiments, the curved cover body 821 abuts against the ternary blades 800 to reduce turbulence. Furthermore, the ternary blades 800 may be connected to the curved cover body 821 by snapping or ultrasonics, or the ternary blades 800 and the curved cover body 821 are integrally formed as a one-piece structure, such that a fully-closed pressurizing centrifugal fan is formed.

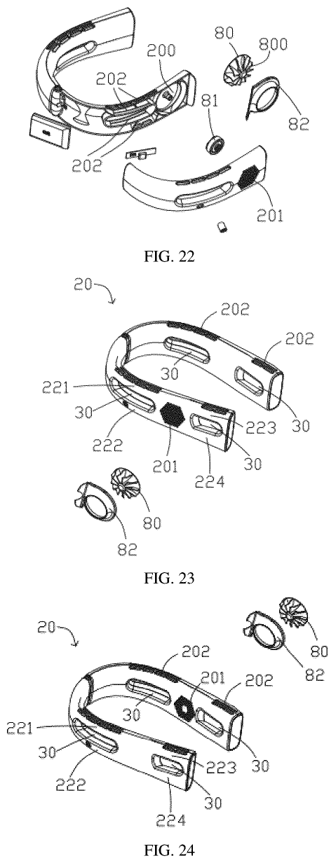

In addition, as shown in FIGS. 21 and 22, in any embodiment herein, the air outlets 202 may be defined in the bridge cover plate 300, which is disposed surrounding the vent holes 30. In some embodiments, the air outlets 202 in the bridge cover plate 300 may be slit-typed air outlets. In other embodiments, the air outlets 202 may be air outletting holes that are spaced apart from each other. To be noted that the air outlets 202 in the bridge cover plate 300 may be inclined towards the neck, enabling the air to flow towards the neck, further improving the ventilation effect, caused by the vent holes 30, on the neck.

To be noted that the ternary blades 800 are arranged in the present embodiment, compared to the centrifugal fan shown in FIGS. 5 to 18, a difference between the radius of the fan blades 80 and the thickness of a rotor shaft is at a centimeter-level. In particular, in the embodiment where the air guiding cover 82 is arranged, the overall size of the fan is larger. The increase in the overall size of the fan increases a size of the steamer-type closed space formed in the body portion and improves the generated heat. In addition, the large sized neck fan is not suitable to be hung around the neck. Therefore, the pressurizing centrifugal fan, which is arranged with the ternary blades 800 as described in the present disclosure, is not arranged in any neck fan in the art. In the present disclosure, by defining the vent holes 30 and arranging the pressurizing centrifugal fan having the ternary blades 800 in combination, an increased size in the pressurizing centrifugal fan having the ternary blades 800 is removed. Therefore, the neck fan is visually lighter, and the increased heat and the steamer-type closed space caused by the large sized blades are avoided, ensuring the user experience and marketability of the neck fan.

It is worth mentioning that, by arranging the pressurizing centrifugal fan having the ternary blades 800, the air inlets 201 of the neck fan in the present disclosure may be located on one side only. For example, the air inlets 201 may be located on the outer side 204 of the neck fan away from the neck. As shown in FIG. 23, which is a schematic view of the neck fan in another embodiment, the fan assembly inside the neck fan is now shown externally. In addition, a plurality of vent holes 30, the first bridge portion 221, the second bridge portion 222, the third bridge portion 223 and the fourth bridge portion 224 are shown.

In other embodiments, the air inlets may alternatively be located on the inner side 203, the top surface or the bottom surface of the body portion 20, as shown in FIG. 24, the air inlets are located on the inner side 203. FIG. 24 shows a schematic view of the neck fan in another embodiment, where the fan assembly inside the neck fan is shown externally. In addition, the plurality of vent holes 30, the first bridge portion 221, the second bridge portion 222, the third bridge portion 223 and the fourth bridge portion 224 are shown.

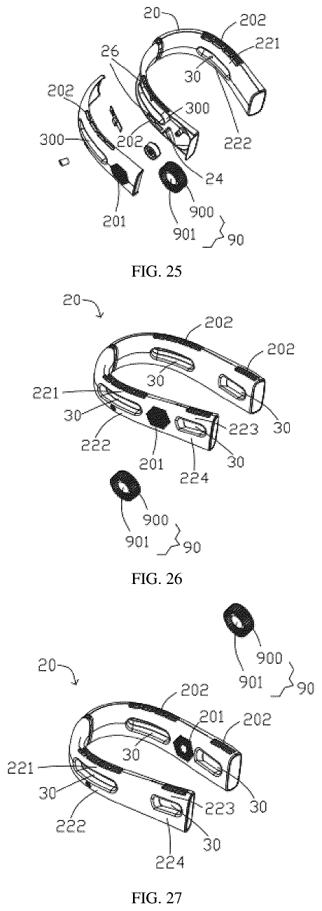

In an embodiment 10, as shown in FIG. 25, the neck fan of the present embodiment has components identical to other embodiments which will not be repeated herein. In the present embodiment, the neck fan further includes a radial single-sided centrifugal fan 90. The radial single-sided centrifugal fan 90 includes a mounting base plate 901 and radial single-sided fan blades 900 connected to the mounting base plate 901. The mounting base plate 901 protrudes towards an air inlet side to form an air guiding platform to increase the airflow pressure.

Accordingly, by arranging the radial single-sided centrifugal fan 90, the air inlets 201 of the neck fan of the present disclosure may be arranged on a single side. For example, the air inlets 201 may be arranged on the outer side 204 away from the neck, or on the inner side 203 adjacent to the neck, or on the top surface or the bottom surface, as shown in FIGS. 26 and 27.

To be added that the radial single-side centrifugal fan 90 shown in FIGS. 25 to 27 differs from the fan blades 103 of the radial double-side centrifugal fan in the previous embodiment in that the fan blades 103 intake air from both sides, whereas the radial single-side centrifugal fan 90 intakes the air from only one side. The fan may be arranged according to various application scenarios, which will not be limited herein.

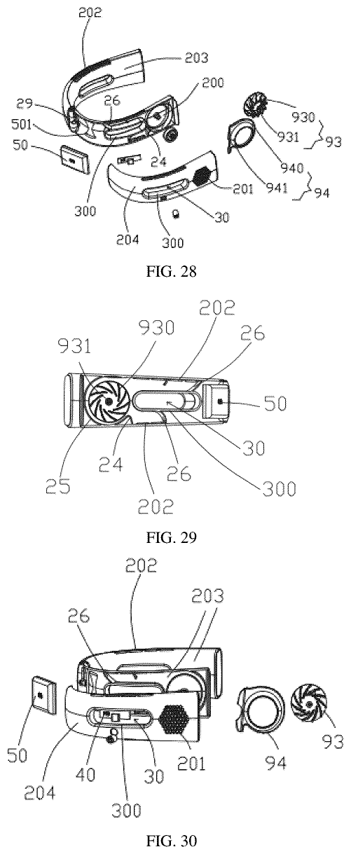

In an embodiment 11, as shown in FIGS. 28 to 32, as previously described, the neck fan includes the fan assembly and the body portion 20. The body portion 20 defines the air inlets 201 and the air outlets 202 and is arranged with the fan assembly. The fan assembly 20 defines one or more vent holes 30 for air circulation. The fan assembly is configured to intake the air from the air inlets 201 and drives the air to flow out of the neck fan from the air outlets 202. The vent holes 30 are configured to allow the air adjacent to the neck to flow freely. The body portion 20 includes the inner side 203 adjacent to the neck and the outer side 204 away from the neck. Each vent hole 30 extends through the inner side 203 and the outer side 204 to allow the air at the outer side 204 to circulate with the air adjacent to the neck.

In the present embodiment, the fan assembly includes a pressurizing centrifugal fan. The pressurizing centrifugal fan includes a motor (not shown in the drawings) and fan blades 93. The fan blades 93 are binary blades. The binary blades may be diagonal binary blades. Specifically, the fan blades 93 includes a mounting base plate 931 and the binary blades 930 arranged on the mounting base plate 931. The binary blades 930 may be fixed to the mounting base plate 931 by snapping or ultrasonics, or the binary blades 930 and the mounting base plate 931 are integrally formed as a one-piece structure. In some embodiments, the binary blades 930 and the mounting base plate 931 may be made of metal, if the cost permits, to enhance a pass rate of finished products.

It is to be added that the diagonal binary blades are arranged in the present embodiment, and unlike the ternary blades, each binary blade is in a form of a sheet. That is, each binary blade is extending on a same plane or a curved surface and does not have a portion twisted and extending from a certain plane. Specifically, as shown in FIG. 29, the binary blades 930 are in overall perpendicular to a plane on which the mounting base plate 931 is arranged. Therefore, a difficulty of molding is reduced, and the number of molds is reduced. Therefore, a production capacity is increased significantly, a mass manufacturing is achievable, the production costs and labor costs are reduced.

As shown in FIGS. 28 and 29, a wind guiding cone may be arranged on a middle portion of a side of the mounting base plate 931 facing the air inlet side to improve the airflow pressure.

Further, the pressurizing centrifugal fan further includes an air guiding cover 94. The air guiding cover 94 is disposed opposite to the fan blades 93 and corresponding to the air inlets 201. The air guiding cover 94 includes a cover plate 940 and a curved cover body 941. The curved cover body 941 is formed by extending from the cover plate 940. An inner surface of the curved cover body 941 corresponding to the binary blades 930 is adapted with an outer contour of the binary blades 930, such that the airflow pressure is increased. In some embodiments, the curved cover body 941 abuts against the binary blades 930 to reduce turbulence. Furthermore, the binary blades 930 may be connected to the curved cover body 941 by snapping or ultrasonics, or the binary blades 930 and the curved cover body 941 are integrally formed as a one-piece structure, such that a fully-closed pressurizing centrifugal fan is formed.

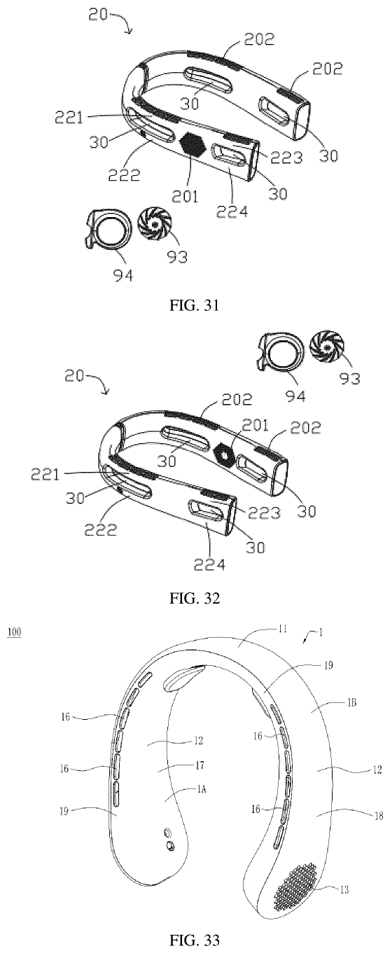

It is worth mentioning that, by arranging the pressurizing centrifugal fan having the binary blades 930, the air inlets 201 of the neck fan in the present disclosure may be located on one side only. For example, the air inlets 201 may be located on the outer side 204 of the neck fan away from the neck. As shown in FIG. 31, which is a schematic view of the neck fan in another embodiment, the fan assembly inside the neck fan is now shown externally. In addition, a plurality of vent holes 30, the first bridge portion 221, the second bridge portion 222, the third bridge portion 223 and the fourth bridge portion 224 are shown.

In other embodiments, the air inlets may alternatively be located on the inner side 203, the top surface or the bottom surface of the body portion 20, as shown in FIG. 32, the air inlets are located on the inner side 203. FIG. 32 shows a schematic view of the neck fan in another embodiment, where the fan assembly inside the neck fan is shown externally. In addition, the plurality of vent holes 30, the first bridge portion 221, the second bridge portion 222, the third bridge portion 223 and the fourth bridge portion 224 are shown.

In addition, as shown in FIG. 28, in the present disclosure, a battery mounting seat 501 is arranged inside the body portion 20 for fixing the battery 50, preventing the battery 50 from shaking. In some embodiments, the battery mounting seat 501 is made of a soft material, such as silicone, and may be made of foam or double sided adhesive tape to adhere the battery 50 to a wall inside the body portion 20, such that the battery 50 is prevented from being shaken and loosened when the user wearing the neck fan is walking, such that after-sale problems may be reduced.

In an embodiment 12, as shown in FIG. 6, FIG. 18, FIG. 20, FIG. 21, FIG. 25, and FIG. 28, a bridge cover plate 300 is formed on the inner shell 211 surrounding the vent holes 30, or the bridge cover plate 300 may be formed on the outer shell 212 surrounding the vent holes 30. Alternatively, a portion of the bridge cover plate 300 is formed on the inner shell 211 surrounding the vent holes 30, and the rest portion of the bridge cover plate 300 is formed on the outer shell 212 surrounding the vent holes 30. In this way, when the inner shell 211 and the outer shell 212 are capped to each other, the portion of the bridge cover plate 300 on the inner shell 211 and the rest portion of the bridge cover plate 300 on the outer shell 212 abut against each other to form the vent holes 30.

In some embodiments, when the inner shell 211 and the outer shell 212 are capped to each other, the portion of the bridge cover plate 300 on the inner shell 211 and the rest portion of the bridge cover plate 300 on the outer shell 212 do not abut against each other, such that a slit-shaped air outlet 202 is formed to improve the ventilation effect of the vent hole 30. In addition, the heat dissipation effect of the battery 50 and/or the control circuit board 40 disposed near the vent hole 30 may be improved.

As shown in FIG. 20, the fan blades in the present embodiment may be the ternary blades that are at least partially twisted, and the other components may be referred to the relevant descriptions in FIGS. 5-FIGS. 32 and will not be repeated. In the present embodiment, by defining the vent holes and arranging the pressurizing centrifugal fan having the ternary blades in combination, an increased size in the pressurizing centrifugal fan having the ternary blades is removed. Therefore, the neck fan is visually lighter, and the increased heat and the steamer-type closed space caused by the large sized blades are avoided, ensuring the user experience and marketability of the neck fan.

As shown in FIGS. 33 to 35, a schematic view of a portable wind blowing device 100 arranged with the fan assembly is shown. The portable wind blowing device 100 includes a bracket 1, and a fan assembly 2 arranged inside the bracket 1. The bracket 1 is configured to be worn on the neck of the user. The bracket 1 defines air inlets 13, a receiving cavity 14, an air duct 15, and air outlets 16, all of which are communicated with each other. The bracket 1 includes a middle portion 11 and two clamping arms 12 that are symmetrically disposed on a left side and a right side of the middle portion 11. The two clamping arms 12 are rotatable relative to the middle portion 11, such that a size of an opening between the two clamping arms 12 is adjusted, facilitating the neck fan to be worn to and removed from the user. The fan assembly 2 is received in the receiving cavity 14 and is configured to drive the air to flow from the air inlet 13 into the air duct 15 and to further flow out of the device through the air outlet 16.

Of course, in other embodiments, the portable wind blowing device 100 may alternatively be worn on an arm, the head, the waist or a leg. Correspondingly, the portable wind blowing device may be a neck fan, a cap fan, an arm fan, a waist fan, or a leg fan. In these cases, a size, a configuration, a material, and softness/hardness of the bracket 1 needs to be adjusted, which is understandable to any ordinary skilled person in the art and will not be described herein.

As shown in FIG. 33 to FIG. 35, in the present embodiment, each clamping arm 12 defines a receiving cavity 14 and an air inlet 13 corresponding to the receiving cavity 14. The air duct 15 extends inside both the clamping arm 12 and the middle portion 11. Each of the clamping arm 12 and the middle portion 11 defines the air outlet 16 communicating the air duct 15 to the exterior of the portable wind blowing device 100. Specifically, the bracket 1 defines two air inlet 13 that are symmetrically located and two receiving cavities 14 that are symmetrically located. A plurality of air outlets 16 are defined and are symmetrically distributed on a side wall of the bracket 1. Two air ducts 15 corresponding to the two receiving cavities 14 may be communicated with or separated from each other. Of course, in other embodiments, the bracket 1 defines two receiving cavities 14 that are symmetrically located about the middle portion 11, and defines two air inlets 13 corresponding to the two receiving cavities 14. The two receiving cavities 14 are separated from each other. The two air ducts 15 corresponding to the two receiving cavities 14 are symmetrically distributed. Each air duct 15 extends from the respective receiving cavity 14 to a free end of the respective clamping arm 12. A plurality of air outlets 16 are defined and are symmetrically distributed on the side wall of the bracket 1.

Of course, in other embodiments, shapes of the two clamping arms 12, the air inlets in the two clamping arms 12, the receiving cavities in the two clamping arms 12, the air outlets in the two clamping arms 12, fan assemblies in the two clamping arms 12, a battery in the two clamping arms 12, and the air ducts in the two clamping arms 12 may be arranged in an asymmetrical manner. These structures may be configured according to user preferences, which will not be limited herein.

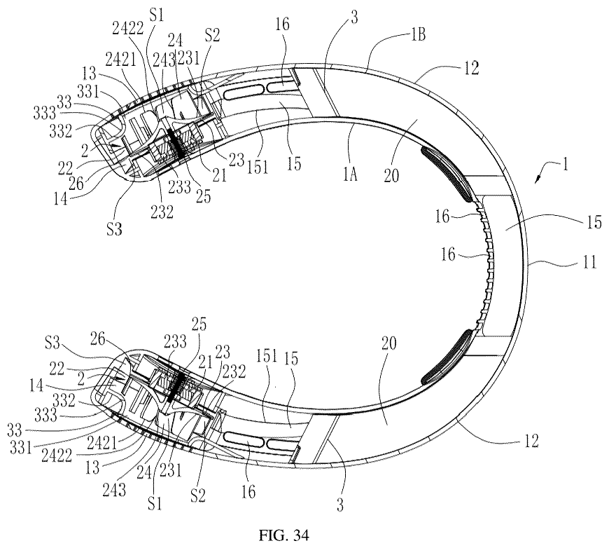

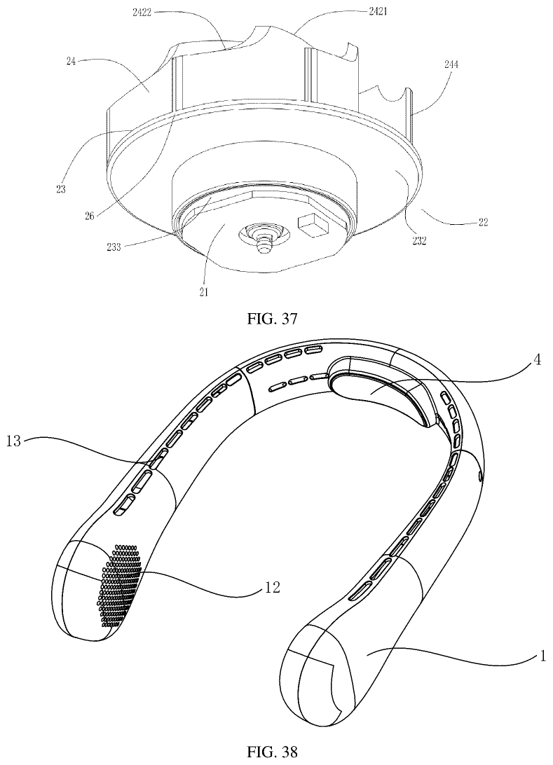

As shown in FIG. 34, FIG. 36, and FIG. 37, the fan assembly 2 includes a motor 21 and fan blades 22. The fan blades 22 are diagonal fan blades. In some embodiments, the fan blades 22 are diagonal centrifugal fan blades. Specifically, the diagonal centrifugal fan blades are binary blades or ternary blades. The diagonal centrifugal fan blades can generate a large airflow volume, which is an advantage of an axial fan, and a large airflow pressure, which is an advantage of a centrifugal fan. Therefore, the diagonal centrifugal fan in the present disclosure has a higher airflow transmission efficiency and a wider range of airflow pressure, greatly improving the air outlet effect. The fan blades 22 include a hub 23 and a plurality of air guiding blades 24. The hub 23 includes a front side 231 facing the air inlets 13 and a rear side 232 facing away from the air inlets 13. The plurality of air guiding blades 24 are evenly arranged on the front side 231 and are spaced apart from each other. The rear side 232 is arranged with a receiving portion 233. A motor 21 is arranged in the receiving portion 233. A diameter of the front side 231 increases in a direction from a center of the front side towards an edge of the front side, such that an airflow guiding cone protruding towards the air inlets 13 is formed. In some embodiments, the front face 231 of the airflow guiding cone is a concave curved surface. That is, a connection from a tip of the airflow guiding cone to a bottom of the airflow guiding cone is a concave curved transition surface. The number of the airflow guiding blades 24 is an odd number. By arranging the odd number of the airflow guiding blades 24, the hub is prevented from having a symmetrical configuration, such that resonance of the hub 23 is prevented, a loss of an airflow speed is prevented.

Of course, in other embodiments, the front face 231 may be a flat surface without the airflow guiding cone, which is not limited herein.

As shown in FIGS. 34, 36 and 37, the hub 23 is arranged with a rotation shaft 25 disposed at a center thereof. Each airflow guiding blade 24 includes a blade bottom portion 241 connected to the hub 23, a blade top portion 242 away from the hub 23, a front edge portion 243 that connects the blade bottom portion 241 to the blade top portion 242 and is disposed near the rotation shaft 25, and a rear edge portion 244 that connects the blade bottom portion 241 to the blade top portion 242 and is disposed away from the rotation shaft 25. The blade bottom portion 241 extends helically on the front face 231. Two blade bottom portions 241 of two fan blades 22 extend in opposite directions on the front face 231. In an axial direction, an air suction channel S1 is formed between inner portions of a plurality of front edge portions 243 in a radial direction and the air inlets 13 and formed between portions of a plurality of blade top portions 242 near the front edge portions 243 and the air inlets 13. In the present embodiment, each blade top portion 242 includes a first blade top portion 2421 near the front edge portion 243 and a second blade top portion 2422 near the rear edge portion 244. That is, in the axial direction, the air suction channel S1 is formed between the inner portions of the plurality of front edge portions 243 in the radial direction and the air inlets 13 and between a plurality of first blade top portions 2421 and the air inlets 13.

As shown in FIGS. 34, 36 and 37, in the present embodiment, each first blade top portion 2421 extends horizontally, and each second blade top portion 2422 extends in an arc shape. Of course, in other embodiments, the first blade top portion 2421 and the second blade top portion 2422 as a whole extend in the arc shape. In the present embodiment, a connection between the first blade top portion 2421 and the front edge portion 243 is arc shaped, facilitating the airflow to be intaken and increasing a contact area between the blade and the air, such that the airflow pressure is increased. A connection between the second blade top portion 2422 and the rear edge portion 244 is a sharp tip, such that the airflow can be cut off, a contact area between the blade and the air is decreased, and a noise is reduced.

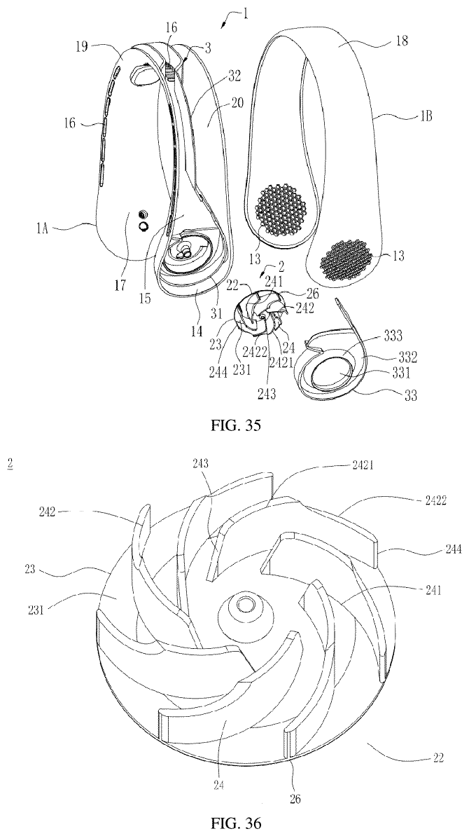

As shown in FIGS. 34 and 35, the portable wind blowing device 100 further includes a partition assembly 3 arranged inside the bracket 1. The partition assembly 3 includes a capacity partition 31 and an air duct partition 32. The capacity partition 31 is configured to define the receiving cavity 14 in the bracket 1, and the air duct partition 32 is configured to define the air duct 15 and a circuit cavity 20 inside the bracket 1. The battery (not shown) and wires (not shown) are received in the circuit cavity 20. The cavity partition 31 and/or the air duct partition 32 may be integrally formed with the bracket 1 as a one-piece structure, or each of the cavity partition 31 and the air duct partition 32 is an independent structure and is assembled into the bracket 1. That is, either one or both of the cavity partition 31 and the air duct partition 32 may be integrally molded with or detachable from the bracket 1, depending on difficulties of molding, costs of molds, difficulties of assembling, and performance of the product, which will not be limited herein.

As shown in FIGS. 34 and 35, in the present embodiment, the portable wind blowing device 100 further includes a cover 33 that is arranged inside the bracket 1 and is located between the blade top portion 242 and the air inlets 13. The cover 33 defines an air suction port 331 corresponding to the air inlet 13. The air suction port 331 exposes the front edge portion 243 and the first blade top portion 2421. A distance between the first blade top portion 2421 and the air inlet 13 is in a range of 4.3 mm to 4.6 mm. A distance between the second blade top portion 2422 and the cover 33 is less than 1.1 mm. In this way, a pressurizing channel S2 is formed between the cover 33 and the front face 231. Of course, in other embodiments, the cover 33 may be omitted, and a body portion of the bracket 1 serves as the cover 33. A distance between the second blade top portion 2422 and the air inlets is in a range of 0.45 mm to 1.1 mm. In this way, the pressurizing channel S2 is formed between the bracket 1 and the front face 231.

To be understood that a portion of the cover 33 serves as the cavity partition 31, and another portion of the cover 33 serves as the air duct partition 32. The cover 33 includes a flat portion 332 and an arc portion 333 extending radially inwardly from the flat portion 332 to protrude towards the air inlet 13. The air suction port 331 is located at an inside of the arc portion 333 in the radial direction. The arc portion 333 is disposed corresponding to the second blade top portion 2422. A distance between the second blade top portion 2422 and the arc portion 333 is less than 1.1 mm.

As shown in FIGS. 34 and 35, an airflow concentration channel S3 is formed between a radial periphery of the air guiding blades 24 and the bracket 1. An air outputting port of the airflow concentration channel S3 correspondingly faces the air duct 15. An angle between the airflow concentration channel S3 and an axis on which the rotation axis 25 is located is greater than or equal to 90° and less than 180°. That is, the airflow concentration channel S3 can be a centrifugal air duct 15 or a diagonal air duct 15. In the airflow concentration channel S3, the airflow is concentrated and then flows into the air duct 15 from the air outputting port of the airflow concentration channel S3. The airflow enters the air suction channel S1 axially from the air inlet 13 and is deflected at an inclined angle through the pressurizing channel S2. In this way, the air that flows from the pressurizing channel S2 to the airflow concentration channel S3 has both an axial component and a radial component. An edge of the hub 23 adjacent to the airflow concentration channel S3 forms a connection portion 26. The connection portion 26 and the rear edge portion 244 cooperatively direct the airflow along a centrifugal direction to the airflow concentration channel S3. An angle between the centrifugal direction and the axis on which the rotation shaft 25 is arranged is greater than or equal to 90° and less than 180°. That is, the airflow is thrown, as a horizontal vortex or as a diagonal vortex, from the pressurizing channel S2 to the airflow concentration channel S3.

In other embodiments, the fan assembly 2 further includes the cavity partition 31 and the cover 33. The cavity partition 31 and the cover 33 cooperatively define the airflow concentration channel S3. In other words, a small sized pressurizing fan assembly, which has the cavity partition 31 and the cover 33 as a shell thereof, is independently formed inside the bracket 1.

As shown in FIGS. 34 and 35, a free end wall of the bracket 1 away from the air inlet 13 is thick, and a bottom of the receiving cavity 14 is recessed. In this way, the rear face 232 of the hub 23 is received, and the motor 21 is shielded by the bottom of the receiving cavity 14 and the receiving portion 233. Therefore, the motor 21 is better protected, and noise of the motor 21 is reduced. A bottom of the airflow concentration channel S3 is higher than the bottom of the receiving cavity 14 and is lower than the connection portion 26. A downward slope portion 151 is formed from the airflow concentration channel S3 towards the middle portion 11. A portion of the covers 33 serves as the air duct partition 32. The downward slope portion 151 and the portion of the cover 33 that serves as the air duct partition 32 cooperatively enables the airflow, which is at an initial stage flowing from the airflow concentration channel S3 towards the air duct 15, remains compressed. In this way, a large airflow pressure is achieved, and a long air delivery path is achieved, and a final air outputting effect is better. Moreover, a space between the bracket 1 and the portion of the cover 33 that serves as the air duct partition 32 may receive a main control module (not shown). The main control module is arranged with a switch of the fan assembly 2, a button (not shown) for adjusting the airflow speed, and a connector (not shown) for connecting to an external power supply. Of course, the main control module may be disposed at a position corresponding to the circuit cavity 20.

As shown in FIGS. 33 to 35, the bracket 1 includes a first shell 1A and a second shell 1B mated with the first shell 1A. In the present embodiment, the receiving cavity 14 is located at the free end of the clamping arm 12. The air inlet 13 is located at the free end of the second shell 1B or at the free end of the first shell 1A. In other embodiments, the air inlet 13 is located in the middle portion 11 of the first shell 1A or in the middle portion 11 of the second shell 1B when the receiving cavity 14 is located in the middle portion 11. In the present embodiment, the first shell 1A includes an inner side wall 17 facing the neck, and the second shell 1B includes an outer side wall 18 facing away from the neck. The first shell 1A or the second shell 1B includes two middle side walls 19 connecting with the inner side wall 17 and the outer side wall 18. The air outlets 16 of the clamping arm 12 is defined in the middle side walls 19. The air outlets 16 of the middle portion 11 are defined in the inner side wall 17 and/or the middle side walls 19. Of course, in other embodiments, the air outlets 16 may be defined at other locations, which are not limited herein.

In combination with the above embodiments and the accompanying drawings, the present disclosure provides a fan assembly configured for the portable wind blowing device. The portable wind blowing device is the portable wind blowing device 100 described in any of the above embodiments. The fan assembly is the fan assembly 2 described in the above embodiments.

As shown in FIGS. 38-48, the portable wind blowing device according to another embodiment of the present disclosure is shown.

As shown in FIGS. 38 to 41, an embodiment 1 of the present disclosure provides a portable wind blowing device. The portable wind blowing device can be hung around and blow wind towards the head, the neck, the arm or the waist. The portable wind blowing device includes a hanging body portion 1 having a receiving cavity 11 and a fan assembly 5 arranged in the receiving cavity 11. The portable wind blowing device takes the hanging body portion 1 to be hung on the neck, and therefore, the portable wind blowing device does not need to be held by hands, the hands of the user are freed. The hanging body portion 1 defines an air inlet 12 and an air outlet 13 that are communicated to the receiving cavity 11. An inner wall of the hanging body portion 1 encloses to form an airflow concentration channel 14 communicated with the air inlet 12 and the air outlet 13. The fan assembly 5 defines an air intaking port at a position corresponding to the air inlet 12. The air intaking port is configured to intake the external air from the air inlet 12 into the receiving cavity 11. The fan assembly 5 drives the air to flow along the airflow concentration channel 14 to the air outlet 13 to be blown out of the device. When the fan assembly 5 is operating, the fan assembly 5 intakes the air from the air inlet 12, pressurizes the air, and drives the air to be blown out of the device from the air outlet 13. In this way, the user is cooled, and the hands of the user are freed. The user may carry the device easily, user demands are met, and the usage experience is improved.

For example, the fan assembly 5 of the present embodiment includes a mixed-flow pressurizing centrifugal blower. The mixed-flow pressurizing centrifugal blower includes a motor for driving. The motor may be a single-phase motor, a three-phase synchronous motor or a three-phase asynchronous motor. The mixed-flow pressurizing centrifugal blower is driven by the three-phase synchronous motor or the three-phase asynchronous motor to rotate at a preset speed to generate a strong negative pressure to intake the external air.

In some embodiments, the three-phase synchronous motor or the three-phase asynchronous motor is arranged. Compared to the single-phase motor, when the device is used for the neck or the head, materials for manufacturing the device is reduced if the three-phase motor (synchronous or asynchronous) is arranged, such that a weight of the device is reduced, and a reduced weight is applied on the neck. Further, the three-phase asynchronous motor of the present embodiment is arranged with a cage rotor. The asynchronous motor having the cage rotor has a simple structure and a reduced weight and can operate reliably, and therefore, production costs are reduced. Of course, a winding three-phase asynchronous motor may be arranged. Correspondingly, the three-phase windings of a rotor and a stator of the winding three-phase asynchronous motor may be arranged and may be connected to an external rheostat by a slip ring and brushes. In this way, a starting performance of the motor may be improved by adjusting a resistance of the rheostat, and the rotation speed of the motor is adjusted by adjusting a resistance of the rheostat.

In the present embodiment, the three-phase synchronous motor or the three-phase asynchronous motor drives the mixed-flow pressurizing centrifugal fan to rotate at the preset rotation speed in a range of 8,000 rpm to 100,000 rpm, or in a range of 12,000 rpm to 75,000 rpm, or in a range of 15,000 rpm to 50,000 rpm, or in a range of 20,000 rpm to 30,000 rpm.

In addition, when the device is hung around the neck, since the device is disposed near the hair and the three-phase motor generally rotates at an ultra-high rotation speed, such as more than 15,000 rpm, a negative pressure effect near the air inlet is very strong. Therefore, in order to preventing sucking the hair into the air inlet 12, a metal mesh cover (such as the mesh cover shown in FIG. 38 and FIG. 39 at air inlet 12, which is not labeled) may be arranged at the air inlet 12 in the present embodiment. In this way, the metal mesh cover blocks foreign matter from entering the three-phase synchronous motor or the three-phase asynchronous motor that is rotating at the high rotation speed. The metal mesh cover itself has certain structural strength and cannot be deformed by compression, such that the metal mesh cover may not touch the three-phase synchronous motor or the three-phase asynchronous motor. To be noted that the metal mesh cover and the hanging body portion 1 may both be made of metal and may be integrally formed as a one-piece structure. In other embodiments, the metal mesh cover may be inlaid in the hanging body portion 1, which is not limited herein.

Correspondingly, for the fan assembly in the present embodiment, the rotation speed of the three-phase synchronous motor or the three-phase asynchronous motor is infinitely adjusted, such that the user can finely and customizedly adjust and balance a wind volume and a wind noise. In this way, the user experience is further improved, and a sudden variation in the rotation speed caused by a multi-gear regulation is prevented, such that a damage to the motor and bearings, caused by the sudden variation in the rotation speed, is prevented, and the service life of the device is extended.

As shown in FIG. 38 to FIG. 40, in the portable wind blowing device, the hanging body portion 1 includes a flexible shell and a shape fixing member fixedly connected to an inner wall of the flexible shell. The receiving cavity 11 is defined in the flexible shell. The shape fixing member is received in the receiving cavity 11 and fixedly connected to the inner wall of the flexible shell. The user can adjust a configuration of the hanging body portion 1 according to usage demands or application scenarios. For example, a bending curvature of the hanging body portion 1 may be adjusted to place the portable wind blowing device around the forehead, the waist, the arm or the leg. Alternatively, the hanging body portion 1 may be twisted to change a facing direction of the air inlet 12 or the air outlet 13, facilitating the user to carry and use the device easily, allowing the device to meet the user's demands. In addition, arranging the flexible shell enables the user to wear the device comfortably, the flexible shell contacts the shoulder and the neck of the user, providing a certain cushion against vibration. A material of the flexible shell of the hanging body portion 1 and a specific configuration of the shape fixing member are not limited herein. In some embodiments, the flexible shell may be made of silicone or thermoplastic polyurethanes (TPU), which provides high strength and toughness. In some embodiments, the shape fixing member may be made by arranging an aluminum strip to an interior of a gooseneck or a serpentine tube, and the entire shape fixing member is fixedly connected to the inner wall of the flexible shell. In this way, the hanging body portion 1 can maintain at a configuration desired by the user, satisfying demands of the user.

As shown in FIG. 38 and FIG. 30, in an embodiment, locations and the number of air inlets 12 and air outlets 13 defined in the hanging body portion 1 of the portable wind blowing device are not limited herein. When the portable wind blowing device is hung around the neck, the air inlet 12 of the hanging body portion 1 may be defined in an inner surface of the hanging body portion 1 facing the neck; or in an outer surface of the hanging body portion 1 facing away from the neck; or in an upper surface of the hanging body portion 1 facing upwardly; or in a lower surface of the hanging body portion 1 facing downwardly. In some embodiments, the air inlet 12 of the hanging body portion 1 is defined in the inner surface of the hanging body portion 1 facing the neck; or in the outer surface of the hanging body portion 1 facing away from the neck. A plurality of air inlets 12 are defined to reduce wind resistance, facilitating the fan assembly 5 to intake the external air to flow through the air inlets 12 to enter the receiving cavity 11, such that the amount of the airflow input into the receiving cavity 11 is ensured, and a power of the airflow that is output out of the device from the air outlet 13 is ensured. In some embodiments, the air inlets 12 on the hanging body portion 1 are defined in the outer surface of the hanging body portion 1 facing away from the neck. The plurality of air inlets 12 are uniformly distributed on the outer surface of the hanging body portion 1. While the amount of the airflow input into the receiving cavity 11 of the hanging body portion 1 is ensured, the noise generated by the operation of the fan assembly 5 is reduced, such that the user experience is improved. In some embodiments, the air outlet 13 of the hanging body portion 1 may be defined in the upper surface of the hanging body portion 1 facing upwardly, such that the airflow generated by the fan assembly 5 in the receiving cavity 11 may flow towards the neck and the head of the user to cool the user. In some embodiments, a plurality of air outlets 13 are defined in the hanging body portion 1. The plurality of air outlets 13 are distributed at equal intervals on the upper surface of the hanging body portion 1 facing upwardly. In this way, the user may cooled by the airflow from various directions, and the user may be cooled rapidly.

In some embodiments, the hanging body portion 1 includes an inner shell and an outer shell that are capped with each other. The inner shell is disposed adjacent to the neck, and the outer shell is disposed away from the neck. The air outlets 13 are defined in the outer shell of the hanging body portion 1 to output the airflow towards the face. Furthermore, the air outlet 13 may be elongated and extend along an extending direction of the hanging body portion 1. An overall curvature of the elongated air outlet 13 is substantially the same as a curvature of the hanging body portion 1, such that the hanging body portion 1 can be processed easily. Since a pathway of the airflow in the airflow concentration channel 14 substantially coincides with the extension of the hanging body portion 1, the elongated air outlet 13 facilitates the airflow in the airflow concentration channel 14 to be blown out of the device, the airflow in the airflow concentration channel 14 is prevented from spinning and forming turbulence, such that the airflow ventilation effect is improved, and the noise is reduced.

In some embodiments, a dustproof sheet is provided on each of the air inlet 12 and the air outlet 13. When the portable wind blowing device is not in use, the dustproof sheet may cover the air inlet 12 and air outlet 13 on the hanging body portion 1, preventing external dust and impurities from entering the receiving cavity 11 from the air inlet 12 and air outlet 13. In this way, the airflow, which is blown out of the receiving cavity 11 when the portable wind blowing device is activated again, is prevented from having the impurities, improving the user experience.

As shown in FIGS. 38 to 39, in the present embodiment 1, the number of fan assemblies 5 arranged in the receiving cavity 11 of the hanging body portion 1 is not limited herein. In some embodiments, two fan assemblies 5 are arranged, and the two fan assemblies 5 are respectively disposed at two ends of the hanging body portion 1. The fan assemblies 5 at the two ends intake air from the exterior of the device and directs the air to flow along the airflow concentration channels 14 on two sides of the hanging body portion 1 to be blown out of the device through the air outlets 13, so as to cool the user.

In some embodiments, one fan assembly 5 is arranged. The fan assembly 5 is disposed in a middle region in a length direction of the hanging body portion 1. The hanging body portion 1 defines at least two air outlets 13. The two air outlet ports 13 are respectively located at opposite sides of the fan assembly 5, enabling the air, which is sucked into the receiving cavity 11 by the fan assembly 5, can be blown out of the device through the two air outlets 13 disposed on the opposite sides of the hanging body portion 1. In this way, the user may be cooled from various directions and may be cooled rapidly.

In some embodiments, three fan assemblies 5 are arranged. Two of the three fan assemblies 5 are respectively disposed at the two ends of the hanging body portion 1, and the rest fan assembly 5 is disposed at the middle region of the hanging body portion 1. The hanging body portion 1 defines at least two air outlets 13. The at least two air outlets 13 are respectively distributed on two opposite sides of the fan assembly 5 at the middle region of the hanging body portion 1. In this way, the air, which is sucked into the receiving cavity 11 by the fan assemblies 5, can be blown out of the device through the two air outlets 13 disposed on the opposite sides of the hanging body portion 1. In this way, the user may be cooled from various directions. In addition, the amount of the air sucked into the portable wind blowing device is effectively increased, such that a power of the airflow output from the air outlets 13 is increased, satisfying the user demands, and improving the usage experience.

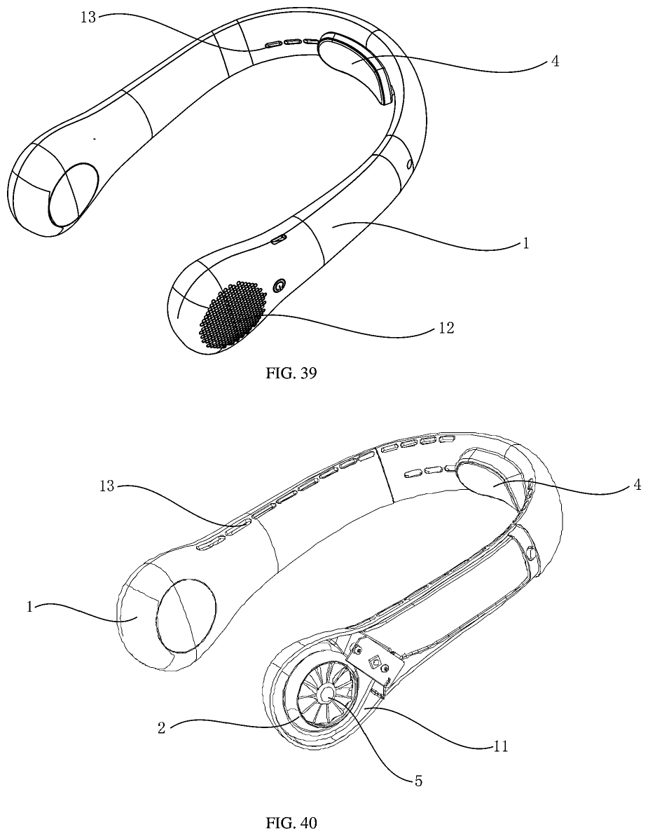

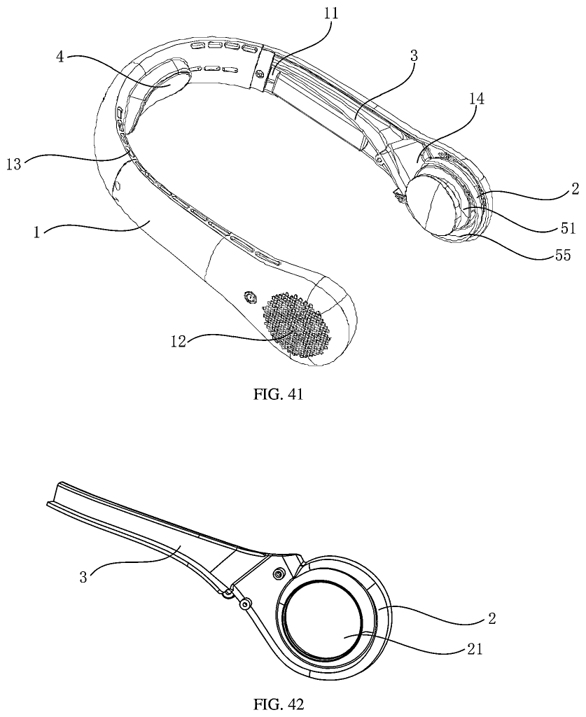

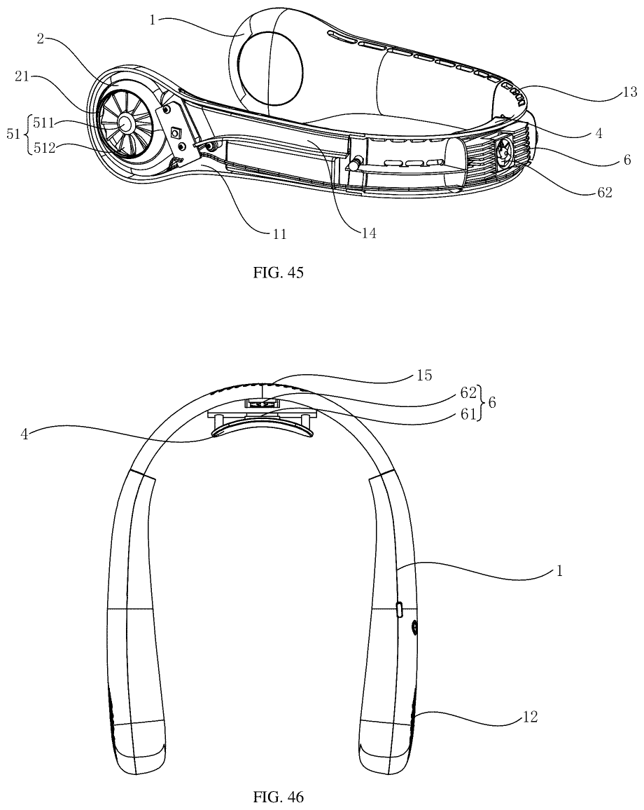

As shown in FIG. 40 and FIG. 45, in an embodiment, the fan assembly 5 includes a mixed-flow pressurizing centrifugal blower 51. The mixed-flow pressurizing centrifugal blower 51 has a high efficiency, low noise, and a compact structure, and can be easily assembled. The mixed-flow pressurizing centrifugal blower 51 includes: an airflow guiding cone 511; a plurality of diagonal fan blades 512, which are disposed at equal intervals along a circumferential outer surface of the airflow guiding cone 511; and a motor configured to drive the airflow guiding cone 511. The motor drives the airflow guiding cone 511 to rotate and drives the plurality of diagonal fan blades 512 disposed on the airflow guiding cone 511 to rotate relative to the motor, taking the airflow guiding cone 511 as a rotation center, to intake the external air into the receiving cavity 11 from the air inlet 12. In addition, an airflow guiding structure 513 is arranged on a side of the diagonal fan blades 512 away from the air inlet to guide a flowing direction of the air. In this way, a centrifugal airflow zone 52 is formed at an outer periphery of the fan assembly 5. The airflow concentration channel 14 is communicated with the centrifugal airflow zone 52. Since the flowing direction of air on the diagonal fan blades 512 is directed by the airflow guiding structure 513, the air, which is intaken from the exterior of the device into the receiving cavity 11, is directed to flow along an extending direction of the airflow guiding structure 513 towards the receiving cavity 11, such that the centrifugal airflow zone 52 is formed at the outer periphery of the fan assembly 5 and in the receiving cavity 11. That is, the flowing direction of the air, which is intaken into the receiving cavity 11 from the air inlet 12, may be regarded as being parallel to a rotation axis of the motor. Under an effect of the airflow guiding structure 513, an angle between a direction that the air is flowing towards the centrifugal airflow zone 52 and a direction that the air is being intaken through the air inlet 12 is in a range of 60 degrees to 120 degrees. That is, the angle between the direction that the air is flowing towards the centrifugal airflow zone 52 and the direction that the air is being intaken through the air inlet 12 is greater than or equal to 60 degrees and is less than or equal to 120 degrees. In other words, an angle between a direction that the airflow concentration channel 14 extends toward the air outlet 13 and a central axis of the airflow guiding cone 511 is in a range of 60 degrees to 120 degrees. That is, the angle between the direction that the airflow concentration channel 14 extends toward the air outlet 13 and the central axis of the airflow guiding cone 511 is greater than or equal to 60 degrees and is less than or equal to 120 degrees. In this way, the airflow is pressurized while being directed, such that the power of the airflow output from the air outlet 13 is increased, meeting demands of the user. In some embodiments, the angle between the direction that the airflow concentration channel 14 extends toward the air outlet 13 and the central axis of the airflow guiding cone 511 is in a range of 80 degrees to 100 degrees. That is, the angle between the direction that the airflow concentration channel 14 extends toward the air outlet 13 and the central axis of the airflow guiding cone 511 is greater than or equal to 80 degrees and is less than or equal to 100 degrees, such as 80 degrees, 81 degrees, 82 degrees, 83 degrees, 84 degrees, 85 degrees, 86 degrees, 87 degrees, 88 degrees, 89 degrees, 90 degrees, 91 degrees, 92 degrees, 93 degrees, 94 degrees or 95 degrees. It is ensured that, when the mixed-flow pressurizing centrifugal blower 51 is intaking and directing the external air, the mixed-flow pressurizing centrifugal blower 51 pressurize the airflow, and the airflow in the centrifugal airflow zone 52 is directed to the air outlet 13 through the airflow concentration channel 14. In this way, the power of the airflow output from the air outlet 13 is improved, the user can be cooled from various directions and can be cooled rapidly, improving the user experience.

In some embodiments, a distance between the fan assembly 5 and the inner wall of the hanging body portion 1 is in a range of 0.5 mm to 1.5 mm. That is, the distance between the fan assembly 5 and the inner wall of the hanging body portion 1 is greater than or equal to 0.5 mm and less than or equal to 1.5 mm, such that a pressurizing channel is formed. The diagonal fan blades on the mixed-flow pressurizing centrifugal blower 51 is prevented from idlely rotating due to the distance between the fan assembly 5 and the inner wall of the hanging body portion 1 being excessive large, such that the intaken airflow is prevented from forming the turbulence, the airflow guiding and pressurizing effect can be achieved. In some embodiments, the distance between the fan assembly 5 and the inner wall of the hanging body portion 1 is 0.5 mm. In this way, the fan assembly 5 can guide and pressurize the airflow, and at the same time, the fan assembly 5 is prevented from abutting against and scratching the inner wall of the hanging body portion 1 due to vibration generated by operation, such that damages to the fan assembly 5 and the hanging body portion 1 are prevented, the service life of the portable wind blowing device can be extended, and the noise generated during the operation is reduced. In other words, the inner wall of the hanging body portion 1 can concentrate the airflow, and therefore, the number of detachable components is reduced, and costs for assembling are reduced.

In some embodiments, the fan assembly 5 further includes an airflow converging shell that sleeves a periphery of the mixed-flow pressurizing centrifugal blower 51. The airflow converging shell defines a convergence channel communicating with the centrifugal airflow zone 52 and the airflow concentration channel 14. When the fan assembly 5 is operating, the external air is intaken into the device, pressurized by the mixed-flow pressurizing centrifugal blower 51, and is guided to the centrifugal airflow zone 52 by the mixed-flow pressurizing centrifugal blower 51. Further, the airflow in the centrifugal airflow zone 52 is then concentrated and guided to the airflow concentration channel 14 through the convergence channel in the airflow converging shell. The airflow concentration channel 14 is communicated with the air outlet 13, enabling the airflow output from the fan assembly 5 to be converged and accelerated through the convergence channel and the airflow concentration channel 14 to further be blown out of the device through the air outlet 13. In this way, a speed of the air flowing out of the air outlet is increased, the power of the airflow output from the air outlet 13 is improved, enabling the user to be cooled rapidly.

In some embodiments, the fan assembly 5 further includes an airflow converging shell that sleeves a periphery of the mixed-flow pressurizing centrifugal blower 51. The airflow converging shell defines a convergence channel communicating with the centrifugal airflow zone 52 and the air outlet 13. When the fan assembly 5 is operating, the external air is intaken into the device, pressurized by the mixed-flow pressurizing centrifugal blower 51, and is guided to the centrifugal airflow zone 52 by the mixed-flow pressurizing centrifugal blower 51. Further, the airflow in the centrifugal airflow zone 52 is then concentrated and guided to the air outlet 13 through the convergence channel in the airflow converging shell. The airflow output from the fan assembly 5 is converged and accelerated by the convergence channel, and then blown out of the device through the air outlet 13. In this way, the speed of the air flowing out of the air outlet is increased, the power of the airflow output from the air outlet 13 is improved, enabling the user to be cooled rapidly.

In some embodiments, the mixed-flow pressurizing centrifugal blower 51 may be a coaxial double-sided mixed-flow blower. The air inlets 12 are defined in two opposite sides of the hanging body portion 1. The mixed-flow pressurizing centrifugal blower 51 intakes the external air from the air inlets 12 located on the two opposite sides of the hanging body portion 1 to enter the receiving cavity 11. In this way, the amount of the external air intaken into the device is increased, the power of the airflow output from the air outlet 13 is improved.

As shown in FIGS. 41 to 43, in the present embodiment 1, the portable wind blowing device further includes an airflow concentration cover 2 received in the receiving cavity 11. The airflow concentration cover 2 and the inner wall of the hanging body portion 1 enclose to cooperatively define form an airflow concentration cavity 55. The fan assembly 5 is mounted in the airflow concentration cavity 55. The airflow concentration cover 2 defines an avoidance port 21 communicating with the airflow concentration cavity 55 and the air inlet 12. Therefore, when the fan assembly 5 is operating, the air flows through the air inlet 12 to enter the avoidance port 21 and further enter the fan assembly 5. The fan assembly 5 starts operating and takes the avoidance port 21 on the airflow concentration cover 2 to direct the external air to flow into air intaking port through the air inlet 12. The fan assembly 5 pressurizes the air and directs the air to the airflow concentration cavity 55. The airflow generated by the fan assembly 5 converges in the airflow concentration cavity 55 and is pressurized again. The pressurized airflow is accelerated by the airflow concentration channel 14 and subsequently flows out of the device through the air outlet 13. In this way, the power of the airflow output from the air outlet 13 is improved, cooling the user rapidly. In other words, in the present embodiment, in order to ensure unity of an outer surface of the hanging body portion 1, the airflow concentration cover 2 is additionally disposed between the inner wall of the hanging body portion 1 and the fan assembly 5. In this way, while performance of the wind blowing device is ensured, a certain cost is increased to improve competitiveness of the product.

To be noted that the airflow concentration channel 14 and the airflow concentration cavity 55 in the present embodiment are structurally or spatially divided from each other. For example, in some embodiments, the airflow concentration channel 14 and the airflow concentration cavity 55 may be equivalent to each other, i.e., the airflow concentration channel 14 is the airflow concentration cavity 55, or in other embodiments, the airflow concentration cavity 55 is a portion of the airflow concentration channel 14.

In some embodiments, the distance between the fan assembly 5 and an inner wall of the airflow concentration cover 2 is in a range of 0.5 mm to 1.5 mm, i.e., the distance between the fan assembly 5 and an inner wall of the airflow concentration cover 2 is greater than or equal to 0.5 mm and is less than or equal to 1.5 mm, such that the pressurizing channel is formed, preventing the diagonal fan blades 512 on the mixed-flow pressurizing centrifugal blower 51 from idlely operating due to the distance between the fan assembly 5 and the inner wall of the airflow concentration cover 2 being excessively large. In this way, the airflow is not turbulence, the air guiding and pressurizing effect are not affected. In some embodiments, the distance between the fan assembly 5 and the inner wall of the airflow concentration cover 2 is 0.5 mm. It is ensured that the fan assembly 5 guides and pressurizes the airflow, and at the same time, the fan assembly 5 is prevented from abutting against and scratching the inner wall of the airflow concentration cover 2 due to vibration generated by operation, such that damages to the fan assembly 5 and the airflow concentration cover 2 are prevented, the service life of the portable wind blowing device can be extended, and the noise generated during the operation is reduced.

In some embodiments, a cushioning member is arranged on an outer surface of the airflow concentration cover 2 and abuts against the inner wall of the hanging body portion 1. The cushioning member is configured to cushion vibration generated when the fan assembly 5 is vibrating. In addition, the cushioning member is configured to reduce noise generated when the airflow concentration cover 2 and the inner wall of the hanging body portion 1 abut against and collide with each other. In some embodiments, the cushioning member is anti-vibration foam or silicone fixedly connected to the outer surface of the airflow concentration cover 2.

As shown in FIG. 41 and FIG. 42, the portable wind blowing device further includes an airflow guiding structure 3 fixedly connected to the airflow concentration cover 2. The airflow guiding structure 3 and the inner wall of the hanging body portion 1 enclose to cooperatively define the airflow concentration channel 14 communicating with the airflow concentration cavity 55 and the air outlet 13. In this way, the airflow output from the fan assembly 5 is accelerated and pressurized by the airflow concentration channel 14 and then is blown out of the device through the air outlet 13, such that the airflow velocity at the air outlet is increased. The airflow guiding structure 513 arranged on the diagonal fan blades 512 of the mixed-flow pressurizing centrifugal blower 51 guides the flowing direction of the airflow, and therefore, the airflow, which is intaken into the airflow concentration cavity 55 from the outside, is guided to flow towards the airflow concentration cavity 55 along the extending direction of the airflow guiding structure 513. In this way, the centrifugal airflow zone 52 is formed at the periphery of the fan assembly 5 and in the airflow concentration cavity 55. That is, the flowing direction of the air, which is intaken into the airflow concentration cavity 55 from the air inlet 12, may be regarded as being parallel to the rotation axis of the motor. Under the effect of the airflow guiding structure 513, an angle between a direction that the air is flowing towards the centrifugal airflow zone 52 and a direction that the air is being intaken through the air inlet 12 is in a range of 60 degrees to 120 degrees. That is, the angle between the direction that the air is flowing towards the centrifugal airflow zone 52 and the direction that the air is being intaken through the air inlet 12 is greater than or equal to 60 degrees and is less than or equal to 120 degrees. In other words, an angle between a direction that the airflow concentration cavity 55 extends towards the air outlet 13 and the central axis of the airflow guiding cone 511 is in a range of 60 degrees to 120 degrees. That is, the angle between the direction that the airflow concentration cavity 55 extends toward the air outlet 13 and the central axis of the airflow guiding cone 511 is greater than or equal to 60 degrees and is less than or equal to 120 degrees. In this way, the airflow is pressurized while being directed, such that the power of the airflow output from the air outlet 13 is increased, and the user can be cooled rapidly.

As shown in FIG. 41 and FIG. 48, in an embodiment, a compression limiting channel 53 is defined between the centrifugal airflow zone 52 and the airflow concentration channel 14. A cross-sectional area of the compression limiting channel 53 is gradually reduced from the centrifugal airflow zone 52 to the airflow concentration channel 14, such that the airflow can be smoothly guided from the centrifugal airflow zone 52 to the airflow concentration channel 14. Since the cross-sectional area of the compression limiting channel 53 is gradually decreased, the pressure and the speed of the airflow increases when the airflow passes through the compression limiting channel 53. Therefore, the airflow, which has been pressurized by the fan assembly 5, enters the centrifugal airflow zone 52 and is concentrated and pressurized therein. The airflow is then pressurized and accelerated by the compression limiting channel 53. While the power of the airflow is increased, the airflow that enters the airflow concentration channel 14 is prevented from flowing reversely to the centrifugal airflow zone 52. In this way, the portable wind blowing device can continuously output the high-pressure airflow to cool the user, meeting the user's demands.

In some embodiments, in another embodiment, the compression limiting channel 53 is formed between the centrifugal airflow zone 52 and the airflow concentration channel 14. The cross-sectional area of the compression limiting channel 53 is smaller than a cross-sectional area of an air outputting portion of the centrifugal airflow zone 52 and is smaller than a cross-sectional area of an air inlet portion of the airflow concentration channel 14. In this way, the airflow can be smoothly guided from the centrifugal airflow zone 52 to the airflow concentration channel 14. Since the cross-sectional area of the compression limiting channel 53 is smaller than the cross-sectional area of the air outputting portion of the centrifugal airflow zone 52 and is smaller than the cross-sectional area of the air inlet portion of the airflow concentration channel 14, the pressure and the speed of the airflow increases when the airflow passes through the compression limiting channel 53. Therefore, the airflow, which has been pressurized by the fan assembly 5, enters the centrifugal airflow zone 52 and is concentrated and pressurized therein. The airflow is then pressurized and accelerated by the compression limiting channel 53. While the power of the airflow is increased, the airflow that enters the airflow concentration channel 14 is prevented from flowing reversely to the centrifugal airflow zone 52. In this way, the portable wind blowing device can continuously output the high-pressure airflow to cool the user, meeting the user's demands.

As shown in FIG. 44 to FIG. 46, the inner surface of a middle region of the hanging body portion 1 is arranged with a support portion 4 protruding from the inner surface. When the device is in use, the hanging body portion 1 is hung on the neck, the support portion 4 separates the inner surface of the hanging body portion 1 from the neck, reducing an area that the hanging body portion 1 contacts the neck. In addition, the inner surface of the middle region of the hanging body portion 1 defines the air outlet 13. Since the support portion 4 separates the inner surface of the hanging body portion 1 from the neck, the air outlet 13 on the inner surface of the hanging body portion 1 is prevented from being blocked by the neck, such that the neck can be cooled optimally.

In some embodiments, the portable wind blowing device further includes the EMS module, configured to generate microcurrents to act on the neck. The EMS module stimulates and massages neck muscles by transmitting mild and gentle electric waves to pass through the epidermis layer to reach the dermis layer of the neck, currents having low intensity and specific waveforms are generated to simulate the cerebrum, the hypothalamus, the limbic reticular formation, such that excitability of the brain can be regulated, insomnia and anxiety may be treated, improving the usage experience. In some embodiments, the EMS module is arranged inside the hanging body portion 1 and is located at the support portion 4, such that the device can be assembled easily, and integrity of the device is improved.

Further as shown in FIG. 44 to FIG. 46, the portable wind blowing device further includes the semiconductor refrigeration module 6. When a direct current provided by a power supply source passes through an electro-couple formed by two different semiconductor materials being connected to each other in series, one of two ends of the electro-couple absorbs heat, and the other one of two ends of the electro-couple releases heat, such that refrigeration is achieved. The semiconductor refrigeration module has a simple structure and is highly reliable. The user may control the semiconductor refrigeration module 6 according to the user's own needs. When the airflow generated by the fan assembly 5 flows through the semiconductor refrigeration module 6, the semiconductor refrigeration module 6 refrigerates the airflow, a temperature range of the airflow blown out of the portable wind blowing device is effectively increased, improving the user experience.

In some embodiments, the semiconductor refrigeration module 6 includes a refrigeration member 61 and a cooling fan 62 for removing heat generated by the refrigeration member 61. The hanging body portion 1 defines a heat dissipation hole 15 at a position corresponding to the cooling fan 62. Refrigeration is achieved by the refrigeration member 61 arranged in the hanging body portion 1, and heat generated by the refrigeration member 61 is dissipated out of the hanging body portion 1 by the cooling fan 62 through the cooling hole 15 defined in the hanging body portion 1. The heat generated by the refrigeration member 61 is prevented from accumulating in the receiving cavity 11 and being mixed in the air flowing through the receiving cavity 11 to be blown out of the hanging body portion 1 through the air outlet 13. Therefore, a temperature of the airflow output from the air outlet 13 is not high, and the user can be cooled effectively. In addition, a localized high temperature is prevented in the hanging body portion 1, such that components inside the hanging body portion 1 are prevented from being deteriorated by the high temperature, and the service life of the portable wind blowing device is extended.

In some embodiments, the fan assembly 5 defines a heat dissipation branched duct for dissipating the heat generated by the refrigeration member 61 of the semiconductor refrigeration module 6. The hanging body portion 1 defines the heat dissipation hole 15 at a position corresponding to the semiconductor refrigeration module 6. Refrigeration is achieved by the refrigeration member 61 in the hanging body portion 1. The heat generated by the refrigeration member 61 is dissipated in time by passing through the heat dissipation branched duct of the fan assembly 5 to be dissipated out of the hanging body portion 1 through the heat dissipation hole 15 defined in the hanging body portion 1. The heat generated by the refrigeration member 61 is prevented from gathering in the receiving cavity 11 and being mixed in the air flowing through the receiving cavity 11 to be blown out of the hanging body portion 1 through the air outlet 13. Therefore, a temperature of the airflow output from the air outlet 13 is not high, and the user can be cooled effectively. In addition, a localized high temperature is prevented in the hanging body portion 1, such that components inside the hanging body portion 1 are prevented from being deteriorated by the high temperature, and the service life of the portable wind blowing device is extended.

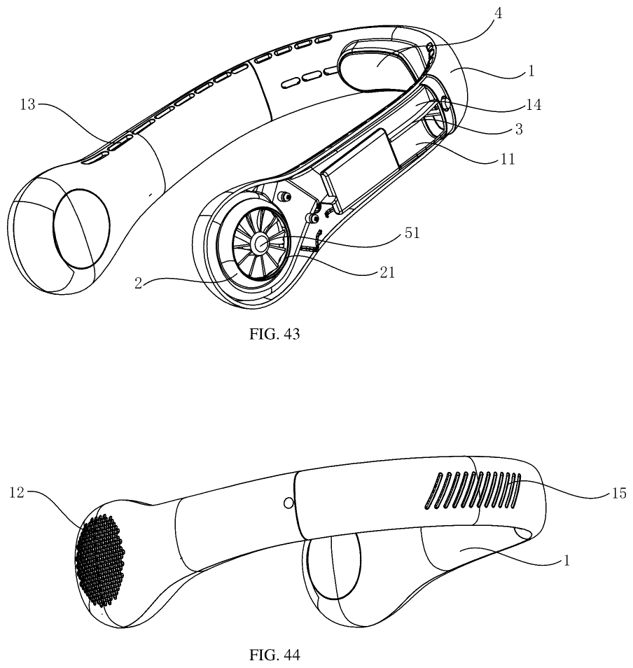

As shown in FIG. 41 and FIG. 43, the portable wind blowing device further includes a power supply source to supply power for the fan assembly 5, the EMS module, and the semiconductor refrigeration module 6. The power supply source may be arranged in the receiving cavity 11 of the hanging body portion 1. Alternatively, the device may be plugged to an external power supply source, such that a size and a weight of the portable wind blowing device is effectively reduced, the user may carry and use the device easily, the user's demands are met.

In some embodiments, the power supply source is a storage battery arranged in the hanging body portion 1. The inner wall of the hanging body portion 1 is arranged with a clamping plate, and a limit slot is defined between the clamping plate and the inner wall of the hanging body portion 1. The power supply source is received in the limit slot to be fixed. On the one hand, the power supply is prevented from vibrating due to shaking in the receiving cavity 11 when the device is in use, such that the power supply is prevented from colliding with the inner wall of the hanging body portion 1 to generate noise, damages to the power supply source can be prevented. The service life of portable wind blowing device is extended. On the other hand, during assembling, the power supply source may be assembled to a preset position in the receiving cavity 11 accurately and easily. An assembling efficiency is increased, and the portable wind blowing device has a more compact configuration, structural stability and reliability of the portable wind blowing device are improved.

The portable blowing device further includes a charging port arranged on the outer surface of the hanging body portion 1 and electrically connected to the power supply source. The user may charge the storage battery in the hanging body portion 1 through the charging port to ensure that the power supply source stores enough electricity for the portable wind blowing device to operate and meet the user's demands.

The portable wind blowing device further includes a control panel electrically connected to the power supply source and a function button electrically connected to the control panel. The function button is mounted on the hanging body portion 1, the control panel is mounted inside the hanging body portion 1. The user may start and stop the portable wind blowing device or select an operation mode by performing operations on the function button mounted on the hanging body portion 1, such that the user's demands are met, and the device is operated easily.

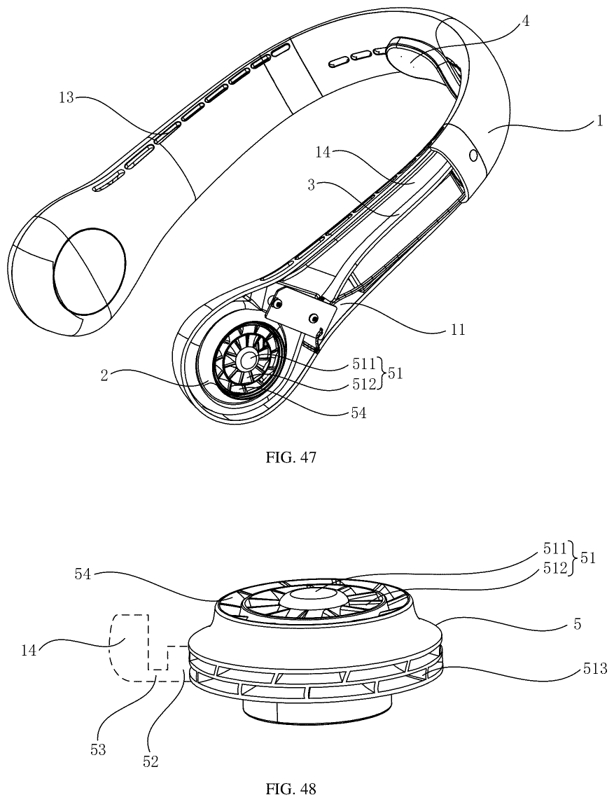

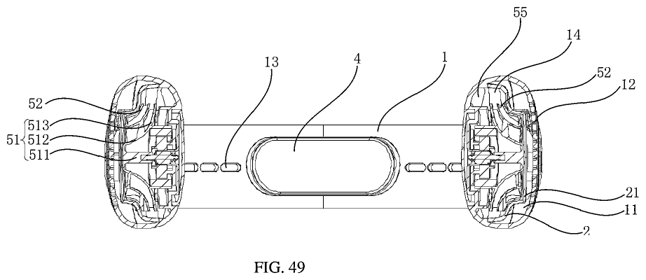

As shown in FIG. 47 to FIG. 49, the fan assembly 5 further includes an auxiliary blower 54 sleeves a periphery of the mixed-flow pressurizing centrifugal blower 51. The motor of the mixed-flow pressurizing centrifugal blower 51 drives the airflow guiding cone 511 to rotate and drives the plurality of diagonal fan blades 512 arranged on the airflow guiding cone 511 to rotate relative to the motor, taking the airflow guiding cone 511 as a rotation center. In this way, the auxiliary fan 54 sleeving the periphery of the mixed-flow pressurizing centrifugal blower 51 is further driven to rotate. The air intaking port includes a main air intaking port communicated with the mixed-flow pressurizing centrifugal blower 51 and an auxiliary air intaking port communicated with the auxiliary fan 54. The mixed-flow pressurizing centrifugal blower 51 and the auxiliary fan 54 cooperatively operate to effectively increase the volume of the external air entering from the air inlet 12 into the receiving cavity 11. In addition, the airflow concentration channel 14 is configured to limit the flowing direction of the air to increase the power of the airflow, such that a high-pressure airflow is increased. The airflow intaken by the mixed-flow pressurizing centrifugal blower 51 and the auxiliary fan 54 is directed by the airflow concentration channel 14 to be blown out of the device from the air outlet 13 to cool the user. In some embodiments, the auxiliary fan 54 may be a direct-flow centrifugal blower or a diagonal-flow centrifugal blower.

In some embodiments, the hanging body 1 and/or the support portion 4 may be sleeved with a removable and replaceable ice material kit (not shown in the drawings) that absorbs moisture and conducts heat. The ice material kit may specifically include cotton threads, ice-sensed fiber threads. The cotton threads and the ice-sensed fiber threads may be arranged crosswisely and braided to form the ice material kit. In the present embodiment, by arranging the removable and replaceable ice material kit, an uncomfortable feeling, which is caused by insufficient heat dissipation and sweat on the neck when the portable wind blowing device is being worn, can be alleviated, ensuring the user to have a cool and comfortable feeling when wearing the portable wind blowing device outdoors.

Of course, in other embodiments, the motor described in the present disclosure may be a single-phase brushless motor having a high rotation speed. In this way, a low-cost single-phase motor replaces the high-cost three-phase motors to enhance market competitiveness of the product.

In some embodiments, the hanging body portion 1 and/or the support portion 4 further defines a battery mounting cavity (not shown in the drawings) in which a backup battery can be inserted or removed, such that batteries can be switchable for use.

In some embodiments, a solar panel may be embedded in the outer shell of the hanging body portion 1, such that the solar panel may generate power by itself when the device is in use outdoors.

The foregoing shows only preferred embodiments of the present disclosure, which do not limit the present disclosure. Any modifications, equivalent substitutions and improvements made within the spirit and principles of the present disclosure shall be included in the scope of the present disclosure.

What is claimed is:

1. A portable wind blowing device, comprising:
a hanging body portion, defining at least one air inlet, a receiving cavity, and at least one air outlet;
at least one fan assembly, received in the receiving cavity;
wherein each of the at least one fan assembly is arranged with:
a centrifugal airflow zone;
a mixed-flow booster blower, wherein the mixed-flow booster blower comprises diagonal fan blades, the diagonal fan blades are configured to direct air from the exterior of the device into the centrifugal airflow zone and to generate a high pressure airflow; and
an airflow concentration channel, configured to concentrate the high pressure airflow in the centrifugal airflow zone and to direct high pressure airflow to be blown out of the device through the at least one air outlet.

2. The portable wind blowing device according to claim 1, wherein, the fan assembly is further arranged with an auxiliary fan, two airflow intaking ports are defined on one side of the fan assembly, and the two airflow intaking ports comprise:
a main airflow intaking port, communicating with the mixed-flow booster blower to form the high pressure airflow; and
an auxiliary airflow intaking port, communicating with the auxiliary fan to form an auxiliary airflow;
wherein, the high pressure airflow and the auxiliary airflow are converged in the airflow concentration channel.

3. The portable wind blowing device according to claim 2, wherein, a compression limiting channel is defined between the centrifugal airflow zone and the airflow concentration channel, a cross-sectional area of the compression limiting channel is smaller than a cross-sectional area of the centrifugal airflow zone;
the portable wind blowing device further comprises an EMS current pulse module, configured to generate a microcurrent to act on the neck;
the portable wind blowing device further comprises a semiconductor refrigeration module, wherein, heat generated by the semiconductor refrigeration module is capable of being dissipated out of the hanging body portion via the high pressure airflow; and
the semiconductor cooling module is integrally arranged with the EMS current pulse module; and/or
the number of the at least one fan assembly is two, the two fan assemblies are respectively located at two ends of the hanging body portion; or the at least one fan assembly is located at a middle of the hanging body portion, the airflow concentration channel has at least two airflow outputting ports that output the airflow towards the two ends of the hanging body portion.

4. A neck fan, comprising:
a fan assembly, comprising a pressurizing centrifugal fan, wherein, the pressurizing centrifugal fan comprises fan blades, the fan blades are diagonal binary blade or ternary blades that are at least partially twisted;
a body portion, defining at least one air inlet and at least one air outlet and receiving the fan assembly;
wherein, the body portion further defines one or more vent holes for air circulation, the fan assembly is configured to intake air through the at least one air inlet and blow the air out of the fan through the at least one air outlet, the one or more vent holes are configured to allow air located adjacent to a neck to flow freely; and/or
the pressurizing centrifugal fan is a mixed-flow pressurizing centrifugal fan, wherein, the mixed-flow pressurizing centrifugal fan comprises a motor for driving; the motor is a three-phase synchronous motor or a three-phase asynchronous motor and is configured to drive the mixed-flow pressurizing centrifugal fan to rotate at a predetermined rotation to generate a strong negative pressure to intake the air from an exterior of the neck fan.

5. The neck fan according to claim 4, wherein, the body portion comprises an inner side adjacent to the neck and an outer side away from the neck; each of the one or more vent holes extends through the inner side and the outer side to allow air at the outer side to circulate with air adjacent to the neck;
wherein, the number of the one or more vent holes is one, and the one vent hole extends along a length direction of the body portion; or the number of the one or more vent holes is two, and the two vent holes are respectively located at a first end and a second end of the body portion; or the number of the one or more vent holes is three, and the three vent holes are respectively located at a first end, a second end, and a middle portion corresponding to a rear of the neck of the body portion; or the number of the one or more vent holes is four or more, and the four or more vent holes are arranged along the length direction of the body portion into an array and are spaced apart from each other.

6. The neck fan according to claim 5, wherein,
the body portion comprises:
an inner shell, comprising an inner shell body, a first inner shell connected to the inner shell body, and a second inner shell connected to the inner shell body; and
an outer shell, comprising an outer shell body, a first outer shell connected to the outer shell body, and a second outer shell connected to the outer shell body;
wherein, the inner shell body and the outer shell body are capped to each other to form a receiving space, the first inner shell and the first outer shell are capped to each other to form a first bridge portion, the second inner shell and the second outer shell are capped to each other to form a second bridge portion, the first bridge portion and the second bridge portion are mated to each other to form the one or more vent holes;
or
the body portion comprises: an inner shell and an outer shell; each of the inner shell and the outer shell is arranged with a bridge cover plate configured to form the one or more vent holes; when the inner shell and the outer shell are capped to each other, a portion of the bridge cover plate formed on the inner shell and another portion of the bridge cover plate formed on the outer shell abut against each other; or a slit is defined between a portion of the bridge cover plate formed on the inner shell and another portion of the bridge cover plate formed on the outer shell, and the slit serves as the at least one air outlet.

7. The neck fan according to claim 6, further comprising a control circuit board, a battery and wires electrically connected to the battery;
wherein, the fan assembly is received in the receiving space; the first bridge portion defines a first air duct and defines the at least one air outlet in an inner, an upper side and/or a lower side of the first bridge portion, and the second bridge portion defines a second air duct and defines the at least one air outlet in an inner, an upper side and/or a lower side of the second bridge portion;
or
wherein, the fan assembly is received in the receiving space; the first bridge portion defines a first air duct and defines the at least one air outlet in an inner, an upper side and/or a lower side of the first bridge portion, and the control circuit board, the battery, and the wires are arranged inside the second bridge portion;
or
the fan, the control circuit board, the battery, and the wires are received in the receiving space; the first bridge portion defines a first air duct and defines the at least one air outlet in an inner, an upper side and/or a lower side of the first bridge portion;
or
one or more fan assemblies are arranged inside the first bridge portion and/or the second bridge portion; and the battery, the control circuit board, and the wires are received in the receiving space;
or
the fan assembly is received in the receiving space; an end of the first bridge portion away from the receiving space and an end of the second bridge portion away from the receiving space are communicated to each other; a semiconductor refrigeration module is arranged inside the second bridge portion; the fan assembly is configured to intake the air from the at least one air inlet and direct the air to flow into the second bridge portion to be cooled/heated by the semiconductor refrigeration module; the fan assembly is further configured to direct the cooled/heated air to flow into the air duct of the first bridge portion through the communicated end and then directs the air to be blown out of the neck fan from the air outlet in the first bridge portion;
or
the fan assembly is received in the receiving space; the first bridge portion defines a first air duct and defines the at least one air outlet in an inner side and/or an upper side of the first bridge portion; a negative-ion generating member is arranged inside the second bridge portion.

8. The neck fan according to claim 6, wherein,
the inner shell further comprises a third inner shell and a fourth inner shell connected to the inner shell body;
the outer shell further comprises a third outer shell and a fourth outer shell connected to the outer shell body;
wherein, the third inner shell and the third outer shell are capped to each other to form a third bridge portion; the fourth inner shell and the fourth outer shell are capped to each other to form a fourth bridge portion; the first bridge portion and the third bridge portion are disposed at different sides of the receiving space, the second bridge portion and the fourth bridge portion are disposed at different sides of the receiving space.

9. The neck fan according to claim 8, wherein,
the fan assembly is received in the receiving space; each of the first bridge portion, the second bridge portion, the third bridge portion, and the fourth bridge portion defines a respective air duct and defines the at least one air outlet in the respective inner side and/or the respective upper side;
or
the fan assembly is received in the receiving space; each of the first bridge portion and the third bridge portion defines a respective air duct and defines the at least one air outlet in the respective inner side and/or the respective upper side, the battery and/or the control circuit board are arranged inside the second bridge portion and/or the fourth bridge portion;
or
the fan assembly is received in the receiving space; each of the first bridge portion and the third bridge portion defines a respective air duct and defines the at least one air outlet in the respective inner side and/or the respective upper side; the end of the first bridge portion away from the receiving space and the end of the second bridge portion away from the receiving space are communicated to each other; an end of the third bridge portion away from the receiving space and an end of the fourth bridge portion away from the receiving space are communicated to each other; the semiconductor refrigeration module is arranged inside the second bridge portion; the fan assembly is configured to intake the air from the at least one air inlet and direct the air to flow into the second bridge portion to be cooled/heated by the semiconductor refrigeration module; the fan assembly is further configured to direct the cooled/heated air to flow into the air duct of the first bridge portion through the communicated end and then directs the air to be blown out of the neck fan from the air outlet in the first bridge portion; the semiconductor refrigeration module is arranged inside the fourth bridge portion; the fan assembly is configured to intake the air from the at least one air inlet and direct the air to flow into the fourth bridge portion to be cooled/heated by the semiconductor refrigeration module; the fan assembly is further configured to direct the cooled/heated air to flow into the air duct of the third bridge portion through the communicated end and then directs the air to be blown out of the neck fan from the air outlet in the fourth bridge portion;
or
the fan assembly is received in the receiving space; the first bridge portion, the second bridge portion, the third bridge portion, and the fourth bridge portion are communicated with each other to form a cooling circulation; the semiconductor refrigeration module is arranged inside each of at least three of the first bridge portion, the second bridge portion, the third bridge portion, and the fourth bridge portion; the fan assembly is configured to intake the air from the at least one air inlet and direct the air to flow into the cooling circulation to be cooled/heated by the semiconductor refrigeration module; the fan assembly is further configured to direct the cooled/heated air to be blown out of the neck fan from the air outlet; the air outlet is located in a last bridge portion of the cooling circulation and/or in an air outputting duct located above and spaced apart from the receiving space;
or
the fan assembly is received in the receiving space; each of the first bridge portion and the third bridge portion defines a respective air duct and defines the at least one air outlet in the respective inner side and/or the respective upper side; and the negative-ion generating member is arranged inside the second bridge portion.

10. The neck fan according to claim 6, further comprising an imitative vortex tongue structure;
wherein, the imitative vortex tongue structure at least partially surrounds the fan assembly; the imitative vortex tongue structure is formed inside the receiving space; or the imitative vortex tongue structure and one or more of the bridge portions are integrally formed and are multiplexed used;
the neck fan further comprises an air duct partition; the air duct partition is formed inside the respective air duct of one or more of the bridge portions to regulate a volume and a pressure of the airflow based on a distance between the air outlet and the fan assembly; and/or
a cross section of a ventilation channel of each vent hole is elongated circular, circular, wavy, or is substantially similar to an overall shape of the body portion.

11. The neck fan according to claim 4, wherein,
the fan blades comprise a mounting base plate and binary blades or ternary blades arranged on the mounting base plate; the binary blades or the ternary blades are fixed to the mounting base plate by snapping or ultrasonics, or the binary blades or the ternary blades and the mounting base plate are integrally formed as a one-piece structure;
the fan blades comprise a mounting base plate and binary blades or ternary blades arranged on the mounting base plate, the binary blades in overall are perpendicular to a plane on which the mounting base plate is arranged;
the fan blades comprise a mounting base plate, and an airflow guiding cone is formed at a middle portion of a side of the mounting base plate facing an air inlet side;
and/or
the three-phase synchronous motor or the three-phase asynchronous motor drives the mixed-flow pressurizing centrifugal fan to rotate at a preset rotation speed in a range of 8,000 rpm to 100,000 rpm, or in a range of 12,000 rpm to 75,000 rpm, or in a range of 15,000 rpm to 50,000 rpm, or in a range of 20,000 rpm to 30,000 rpm to extend an operation time length of the neck fan powered by a battery.

12. The neck fan according to claim 11, wherein,
the pressurizing centrifugal fan further comprises an airflow guiding cover, the airflow guiding cover is disposed opposite to the fan blades and corresponding to the air inlet; the airflow guiding cover comprises a cover plate and a curved cover body; the curved cover body is extending from and molded with the cover plate; a shape of an inner surface of the curved cover body corresponding to the binary blades is adapted to an outer contour of the binary blades;
or
the pressurizing centrifugal fan further comprises an airflow guiding cover, the airflow guiding cover is disposed opposite to the fan blades and corresponding to the air inlet; the airflow guiding cover comprises a cover plate and a curved cover body; the curved cover body abuts against the binary blades.

13. A centrifugal fan assembly, intaking air from a single side and configured in a portable wind blowing device, the centrifugal fan assembly comprising a motor and fan blades, wherein the fan blades comprise a hub and a plurality of airflow guiding blades;
wherein, the hub comprises a front side facing an air inlet of the portable wind blowing device and a rear side facing away from the air inlet of the portable wind blowing device; the plurality of the airflow guiding blades are arranged on and protrude from the front side; each of the plurality of airflow guiding blades comprises a blade top portion facing away from the front side;
an airflow intaking space, through which the air is intaking from the single side, is formed between the front face of the hub and the blade top portion of the airflow guiding blade; and/or an axial vertical height between the blade top portion of the airflow guiding blade and the front side of the hub at least partially varies gradually in a radial direction of the hub.

14. The centrifugal fan assembly according to claim 13, wherein, each airflow guiding blade further comprises a front edge portion connected between the blade top portion and the front side of the hub; and the front edge portion is inclined with respect to the front side of the hub.

15. The centrifugal fan assembly according to claim 14, wherein, the blade top portion of the airflow guiding blade comprises a first top portion disposed near the front edge portion and a second top portion away from the front edge portion; the second top portion is recessed toward the front side of the hub.

16. The centrifugal fan assembly according to claim 15, wherein, the first top portion of the airflow guiding blade extends horizontally.

17. The centrifugal fan assembly according to claim 15, wherein, the second top portion of the airflow guiding blade has a first end connected to the first top portion and a free end, serving as a second end, away from the first top portion; a vertical distance from the first end to the front side of the hub is greater than a vertical distance from the second end to the front side of the hub.

18. The centrifugal fan assembly according to claim 15, wherein, in an axial direction of the hub, an airflow suction channel is formed between a radial inner portion of the front edge portion of the airflow guiding blade vane and the air inlet of the portable wind blowing device and formed between the first top portion of the airflow guiding blade and air inlet of the portable wind blowing device.

19. The centrifugal fan assembly according to claim 18, wherein, a connection portion is formed at an edge of the hub adjacent to the airflow concentration channel, the connection portion and a rear edge portion of the airflow guiding blade are cooperatively configured to direct the air to flow towards the airflow concentration channel along a centrifugal direction; an angle between the centrifugal direction and an axis on which a rotation shaft of the hub is arranged is greater than or equal to 90° and less than 180°.

20. The centrifugal fan assembly according to claim 13, wherein, a diameter of the front side of the hub gradually increases from a center of the hub towards an edge of the hub, and the front side has a concave and curved surface.
